SEQUENCE LISTING (4 sequences)

SEQ ID NO: 1 - DNA, 4107 bp, Artificial sequence
  Codon optimized (wheat) cas9 gene
atggacaagaagtactccatcggcctcgacatcggcaccaacagcgtcggctgggccgtgatcaccgacgagtacaaggtgccaagcaagaagttcaaggtgctgggcaacaccgaccgtcactccatcaagaagaacctcatcggcgccctcctcttcgactcaggtgagacggccgaggcgacaaggttgaagaggacggcaagaagaaggtacacaagaaggaagaacaggatctgctacctccaggagatcttcagcaacgagatggccaaggtggatgacagcttcttccaccggctagaggagagcttcttggtggaggaggacaagaagcatgagaggcatcccatcttcggcaacatcgtcgacgaggtggcctaccatgagaagtaccccaccatctaccatctccgcaagaagctggtggactcaacggacaaggccgacctccgcctcatctacctcgccttggcccacatgatcaagttcagaggccacttcctcatcgagggcgacctcaaccctgacaacagcgatgtggacaagctcttcatccagctggtgcagacctacaaccagctctttgaggagaaccccatcaacgcctccggcgtcgacgccaaggccatcctctcggcaaggttgagcaagtcaagaaggctggagaacctcatcgctcagctccccggcgagaagaaaaatggcctcttcggcaacctcatcgccttgagcctcggcctcacccccaacttcaagtcaaactttgacctcgccgaggatgccaagctccagctctccaaggacacctacgacgacgacctcgacaacctcctcgcccagatcggcgaccagtacgccgacctcttcctcgccgccaagaacctctcagatgccatcctcctgagcgacatcctccgcgtcaacaccgagatcaccaaggcgccgctctccgcctccatgatcaagaggtatgatgagcaccatcaagatctcaccctcctcaaggccttggtgaggcagcagctacctgagaagtacaaggagatcttcttcgaccagagcaagaatggctacgccggctacatcgacggcggcgcctcccaagaagagttctacaagttcatcaagcccatcttggagaagatggatggcaccgaggagctactggtgaagctcaacagagaagatctcctcaggaagcagaggaccttcgacaacggcagcatccctcaccagatccaccttggtgagctccatgccatcttgagaaggcaagaagacttctaccccttcctcaaggacaacagggagaagatcgagaagatattgacattcaggatcccctactacgtcggacccttggcaagaggcaactcaaggttcgcctggatgacaaggaagagcgaggaaaccatcaccccctggaactttgaggaggtggtggacaagggcgcctcagctcagagcttcatcgagaggatgaccaacttcgacaagaacctccccaacgagaaggtgctccccaagcacagcctcctctacgagtacttcaccgtctacaatgagctcaccaaggtcaagtacgtcaccgagggcatgaggaagccggccttcctctcaggagagcagaagaaggccatcgtcgacctcctcttcaagaccaacaggaaggtcaccgtcaagcagctcaaggaggactacttcaagaagatcgagtgcttcgactctgtggagatctccggcgtggaggacaggttcaacgcctccctcggcacctaccatgatctcctgaagatcatcaaggacaaggacttcctcgacaacgaggagaacgaggacatcttagaagacatcgtcctcaccctcaccctcttcgaggacagggagatgatcgaggagaggctcaagacctatgctcacctctttgatgacaaggtgatgaagcagctcaagagaagaaggtacaccggctggggccgcctctcaaggaagctcatcaacggcatcagggacaagcagagcggcaagaccatcctcgacttcctcaagagcgatggcttcgccaacaggaacttcatgcagctcatccatgatgacagcttgaccttcaaggaggacatccagaaggctcaagtctccggccaaggagattccctccatgagcacatcgccaacctcgccggctcaccggccatcaagaagggcatcctccagaccgtcaaggtggtggatgagctggtgaaggtgatgggccgtcacaagccggagaacatcgtcatcgagatggcaagggagaaccagaccacccagaagggccagaagaactcaagggagaggatgaagaggattgaagaaggcatcaaggagctcggcagccagatcctcaaggagcaccctgtggagaacacccagctacagaacgagaagctctacctctactacctccagaatggccgtgacatgtacgtcgaccaagagctggacatcaacaggttgagcgactacgacgtcgaccacatcgtcccccagagcttcctcaaggatgacagcatcgacaacaaggtgctgacaagatcagacaagaaccgtggcaagagcgacaatgtcccctccgaggaggtggtgaagaagatgaagaactactggaggcagctcctcaacgccaagctcatcacccagaggaagttcgacaacctcaccaaggctgaaagaggaggcctctcagagctggacaaggccggcttcatcaagaggcagctggtggagacaaggcagatcaccaagcatgttgctcagatccttgattcaaggatgaacaccaagtacgacgagaacgacaagctcatcagggaggtcaaggtcatcaccctcaagagcaagctggtgtccgacttcaggaaggacttccagttctacaaggtgagggagatcaacaactaccaccatgctcatgatgcctacctcaacgccgtggtgggcaccgccctcatcaagaagtaccccaagctggagtcagagttcgtctacggcgactacaaggtgtatgatgtgaggaagatgatcgccaagagcgagcaggagatcggcaaggccaccgccaagtacttcttctactcaaacatcatgaacttcttcaagaccgagatcaccctcgccaacggcgagatcaggaagaggccgctcattgagacaaatggtgagactggtgagatcgtctgggacaagggccgagacttcgccaccgtgaggaaggtgctctccatgcctcaagtcaacatcgtcaagaagaccgaggtgcagaccggaggcttctcaaaggagagcatcctccccaagaggaactctgacaagctgatcgcaaggaagaaggactgggaccccaagaagtacggcggctttgattcaccaacggtggcctactccgtgctggtggtggccaaggtggagaagggcaagagcaagaagctcaagagcgtcaaggagctcctcggcatcaccatcatggagagatcatccttcgagaagaaccccatcgacttccttgaggccaagggctacaaggaggtgaagaaggacctcatcatcaagctccccaagtacagcctcttcgagctggagaatggccgcaagaggatgctggcatcagctggagagctccagaagggcaacgagctggccctcccaagcaagtacgtgaacttcctctacctcgcctcccactacgagaagctcaagggctcaccagaagacaatgagcagaagcagctcttcgtggagcagcacaagcactacctcgacgagatcatcgagcagatctccgagttctccaagagggtgatcctcgccgacgccaacctcgacaaggtgctctcagcctacaacaagcaccgtgacaagcccatcagggagcaagctgagaacatcatccacctcttcaccctcaccaacctcggcgccccggcggccttcaagtacttcgacaccaccatcgaccgcaagaggtacacctcaaccaaggaggtgcttgatgccaccttgatccaccagagcatcaccggcctctacgagacaaggatcgacctctcccagctcggcggcgactga

SEQ ID NO: 2 - protein, 1368 aa, Artificial sequence [sequence omitted]

SEQ ID NO: 3 - DNA, 4206 bp, Artificial sequence
  Codon optimized (wheat) cas9 gene (with fused NLS)
atggcacccaagaagaagaggaaggtgggcatccacggcgtcccagcagcaatggacaagaagtactccatcggcctcgacatcggcaccaacagcgtcggctgggccgtgatcaccgacgagtacaaggtgccaagcaagaagttcaaggtgctgggcaacaccgaccgtcactccatcaagaagaacctcatcggcgccctcctcttcgactcaggtgagacggccgaggcgacaaggttgaagaggacggcaagaagaaggtacacaagaaggaagaacaggatctgctacctccaggagatcttcagcaacgagatggccaaggtggatgacagcttcttccaccggctagaggagagcttcttggtggaggaggacaagaagcatgagaggcatcccatcttcggcaacatcgtcgacgaggtggcctaccatgagaagtaccccaccatctaccatctccgcaagaagctggtggactcaacggacaaggccgacctccgcctcatctacctcgccttggcccacatgatcaagttcagaggccacttcctcatcgagggcgacctcaaccctgacaacagcgatgtggacaagctcttcatccagctggtgcagacctacaaccagctctttgaggagaaccccatcaacgcctccggcgtcgacgccaaggccatcctctcggcaaggttgagcaagtcaagaaggctggagaacctcatcgctcagctccccggcgagaagaaaaatggcctcttcggcaacctcatcgccttgagcctcggcctcacccccaacttcaagtcaaactttgacctcgccgaggatgccaagctccagctctccaaggacacctacgacgacgacctcgacaacctcctcgcccagatcggcgaccagtacgccgacctcttcctcgccgccaagaacctctcagatgccatcctcctgagcgacatcctccgcgtcaacaccgagatcaccaaggcgccgctctccgcctccatgatcaagaggtatgatgagcaccatcaagatctcaccctcctcaaggccttggtgaggcagcagctacctgagaagtacaaggagatcttcttcgaccagagcaagaatggctacgccggctacatcgacggcggcgcctcccaagaagagttctacaagttcatcaagcccatcttggagaagatggatggcaccgaggagctactggtgaagctcaacagagaagatctcctcaggaagcagaggaccttcgacaacggcagcatccctcaccagatccaccttggtgagctccatgccatcttgagaaggcaagaagacttctaccccttcctcaaggacaacagggagaagatcgagaagatattgacattcaggatcccctactacgtcggacccttggcaagaggcaactcaaggttcgcctggatgacaaggaagagcgaggaaaccatcaccccctggaactttgaggaggtggtggacaagggcgcctcagctcagagcttcatcgagaggatgaccaacttcgacaagaacctccccaacgagaaggtgctccccaagcacagcctcctctacgagtacttcaccgtctacaatgagctcaccaaggtcaagtacgtcaccgagggcatgaggaagccggccttcctctcaggagagcagaagaaggccatcgtcgacctcctcttcaagaccaacaggaaggtcaccgtcaagcagctcaaggaggactacttcaagaagatcgagtgcttcgactctgtggagatctccggcgtggaggacaggttcaacgcctccctcggcacctaccatgatctcctgaagatcatcaaggacaaggacttcctcgacaacgaggagaacgaggacatcttagaagacatcgtcctcaccctcaccctcttcgaggacagggagatgatcgaggagaggctcaagacctatgctcacctctttgatgacaaggtgatgaagcagctcaagagaagaaggtacaccggctggggccgcctctcaaggaagctcatcaacggcatcagggacaagcagagcggcaagaccatcctcgacttcctcaagagcgatggcttcgccaacaggaacttcatgcagctcatccatgatgacagcttgaccttcaaggaggacatccagaaggctcaagtctccggccaaggagattccctccatgagcacatcgccaacctcgccggctcaccggccatcaagaagggcatcctccagaccgtcaaggtggtggatgagctggtgaaggtgatgggccgtcacaagccggagaacatcgtcatcgagatggcaagggagaaccagaccacccagaagggccagaagaactcaagggagaggatgaagaggattgaagaaggcatcaaggagctcggcagccagatcctcaaggagcaccctgtggagaacacccagctacagaacgagaagctctacctctactacctccagaatggccgtgacatgtacgtcgaccaagagctggacatcaacaggttgagcgactacgacgtcgaccacatcgtcccccagagcttcctcaaggatgacagcatcgacaacaaggtgctgacaagatcagacaagaaccgtggcaagagcgacaatgtcccctccgaggaggtggtgaagaagatgaagaactactggaggcagctcctcaacgccaagctcatcacccagaggaagttcgacaacctcaccaaggctgaaagaggaggcctctcagagctggacaaggccggcttcatcaagaggcagctggtggagacaaggcagatcaccaagcatgttgctcagatccttgattcaaggatgaacaccaagtacgacgagaacgacaagctcatcagggaggtcaaggtcatcaccctcaagagcaagctggtgtccgacttcaggaaggacttccagttctacaaggtgagggagatcaacaactaccaccatgctcatgatgcctacctcaacgccgtggtgggcaccgccctcatcaagaagtaccccaagctggagtcagagttcgtctacggcgactacaaggtgtatgatgtgaggaagatgatcgccaagagcgagcaggagatcggcaaggccaccgccaagtacttcttctactcaaacatcatgaacttcttcaagaccgagatcaccctcgccaacggcgagatcaggaagaggccgctcattgagacaaatggtgagactggtgagatcgtctgggacaagggccgagacttcgccaccgtgaggaaggtgctctccatgcctcaagtcaacatcgtcaagaagaccgaggtgcagaccggaggcttctcaaaggagagcatcctccccaagaggaactctgacaagctgatcgcaaggaagaaggactgggaccccaagaagtacggcggctttgattcaccaacggtggcctactccgtgctggtggtggccaaggtggagaagggcaagagcaagaagctcaagagcgtcaaggagctcctcggcatcaccatcatggagagatcatccttcgagaagaaccccatcgacttccttgaggccaagggctacaaggaggtgaagaaggacctcatcatcaagctccccaagtacagcctcttcgagctggagaatggccgcaagaggatgctggcatcagctggagagctccagaagggcaacgagctggccctcccaagcaagtacgtgaacttcctctacctcgcctcccactacgagaagctcaagggctcaccagaagacaatgagcagaagcagctcttcgtggagcagcacaagcactacctcgacgagatcatcgagcagatctccgagttctccaagagggtgatcctcgccgacgccaacctcgacaaggtgctctcagcctacaacaagcaccgtgacaagcccatcagggagcaagctgagaacatcatccacctcttcaccctcaccaacctcggcgccccggcggccttcaagtacttcgacaccaccatcgaccgcaagaggtacacctcaaccaaggaggtgcttgatgccaccttgatccaccagagcatcaccggcctctacgagacaaggatcgacctctcccagctcggcggcgacaagaggccggcggccaccaagaaggccggccaagccaagaagaagaagtga

SEQ ID NO: 4 - protein, 1401 aa, Artificial sequence [sequence omitted]

(19) United States
(12) Patent Application Publication
(10) Pub. No.: US 2023/0075913 A1
(43) Pub. Date: Mar. 9, 2023

(54) CODON-OPTIMIZED CAS9 ENDONUCLEASE ENCODING POLYNUCLEOTIDE

(71) Applicant: BASF Agricultural Solutions Seed US LLC, Florham Park, NJ (US)

(72) Inventors: David DE VLEESSCHAUWER, Ghent (BE); Frank MEULEWAETER, Gent (BE); Timothy James GOLDS, Ghent (BE)

(21) Appl. No.: 17/785,816

(22) PCT Filed: Nov. 30, 2020
(86) PCT No.: PCT/EP2020/083861

§ 371 Date: Jun. 15, 2022

(30) Foreign Application Priority Data
Dec. 16, 2019 (EP) 19216387.1

(51) Int. Cl. C12N 15/52; C12N 15/82; C12N 9/22

(52) U.S. Cl. CPC C12N 15/52; C12N 15/8241; C12N 9/22; C12N 15/8213; C12N 2310/20

(57) ABSTRACT

It was now found that the expression of a nucleotide sequence as described in the method of the invention in a plant cell results in much higher rates of indels compared to those seen in cells transformed with a control nucleic acid molecule. Thus, the invention is directed to codon-optimized Cas9 endonuclease-encoding polynucleotide. Accordingly, the present invention provides a method for modifying a target site in the genome of a plant cell, the method comprising providing one or more guide RNA and a Cas endonuclease to said plant cell, wherein said guide RNA and Cas endonuclease are capable of forming a complex that enables the Cas endonuclease to introduce a double strand break at said target site, and wherein the Cas9 endonuclease is expressed in the plant cell from a polynucleotide comprising an codon-optimized Cas9 endonuclease encoding nucleic acid molecule with a nucleotide sequence selected from the disclosed nucleotide sequences.

### FIELD OF THE INVENTION

The invention is directed to codon-optimized Cas9 endonuclease encoding polynucleotide.

### DESCRIPTION OF THE INVENTION

The CRISPR (clustered regularly interspaced short palindromic repeats) system was initially identified as an adaptive defense mechanism of bacteria belonging to the genus of Streptococcus (WO2007/025097). Those bacterial CRISPR systems rely on guide RNA (gRNA) forming a complex with cleaving proteins to direct degradation of complementary sequences present within invading viral DNA. Cas9, the first identified protein of the CRISPR/Cas system, is a large monomeric DNA nuclease that is guided to a DNA target sequence adjacent to the PAM (protospacer adjacent motif) sequence motif by a complex of two noncoding RNAs: crRNA and trans-activating crRNA (tracrRNA).

This widespread system occurs in nearly half of bacteria (˜46%) and the large majority of archaea (˜90%). They are classified into six main CRISPR-Cas systems types (Makarova et al. 2011. Nature Rev. Microbiol. 9:467-477; Makarova et al. 2015. Nature Rev. Microbiol. 13:722-736) based on the cas gene content, organization and variation in the biochemical processes that drive crRNA biogenesis, and Cas protein complexes that mediate target recognition and cleavage.

Jinek et. al. could show, that a synthetic RNA chimera (single guide RNA or gRNA) created by fusing crRNA with tracrRNA is equally functional (Jinek et. al., 2012).

Several research groups have found that the CRISPR cutting properties could be used to disrupt genes in almost any organism's genome with unprecedented ease (Mali P, et al (2013) Science. 339(6121):819-823; Cong L, et al (2013) Science 339(6121)). Recently, it became clear that a template for repair allowed for editing the genome with nearly any desired sequence at nearly any site, transforming CRISPR into a powerful gene editing tool (WO/2014/150624, WO/2014/204728).

Gene targeting refers to site specific gene modification by nucleic acid deletion, insertion or replacement via homologous recombination (HR). Targeting efficiency is highly promoted by a double-strand break (DSB) in the genomic target. Also, the direct presence of homology after DSB of chromosomal DNA seems to nearly eliminate non-homologous end joining (NHEJ) repair in favor of homologous recombination.

The new approach to genome editing using the CRISPR/Cas9 system has now been applied widely to many plant species including crop plants. Several recent reports describe successful CRISPR/Cas9-mediated targeted mutagenesis in plants (Baltes and Voytas for review).

CRISPR/Cas9 system has been developed in plants such as Arabidopsis, tobacco and rice, including crop species, as maize, wheat and soybean.

Cas9 expression level and mutation frequency are positively correlated (Mikami et al. Plant Mol Biol., 2015; 88(6): 561-572.). Li et al. (Nat Biotechnol. 2013; 31:688-691) showed different Cas9 protein expression levels and different mutation frequency in Arabidopsis protoplasts transformed with a Cas9 gene optimized differently for plants and mammals. Thus, it was assumed, that codon usage of Cas9 affects stability, translation efficiency and/or splicing pattern of mRNA and, probably, the amount of functional Cas9 protein in plant cells.

Recently, Osakabe, Y et al. (2016, Sci. Rep. 6, 26685) described that in a direct comparison of the expression levels of both RNA and protein of Cas9 and GFP in the Arabidopsis plants suggested that mutation rates were not correlated strongly with expression levels; however, the high expression levels of Cas9 were sufficient to direct effective genome editing.

Although several approaches have been developed to improve the efficiency of the Cas9 system in plants, there still remains a need for more efficient and effective methods for producing plants having an altered genome comprising specific modifications in a defined region of the genome of the plant.

It is, thus, one objective of the invention at hand to enhance the efficiency to introduce double strand breaks at a target site in DNA of plant cells, for example in cells of monocots, e.g. in wheat.

This was achieved by identifying a new nucleotide sequence coding for a Cas9 protein that if expressed in a plant cell results in an improved efficiency in double strand break introduction at a target site by the encoded Cas9 endonuclease.

Accordingly, the present invention provides a method for modifying a target site in the genome of a plant cell, the method comprising providing one or more guide RNA and a Cas endonuclease to said plant cell, wherein said guide RNA and Cas endonuclease are capable of forming a complex that enables the Cas endonuclease to introduce a double strand break at said target site, and wherein the Cas9 endonuclease is expressed in the plant cell from a polynucleotide comprising a codon-optimized Cas9 endonuclease encoding nucleic acid molecule with a nucleotide sequence selected from the following nucleotide sequences:
a. a nucleotide sequence that in an alignment with the nucleotide sequence depicted in SEQ ID NO 1 has at the following positions counting from the first nucleotide of the start codon one or more of the following nucleotide combinations:
   i. 3018 A and 201 G,
   ii. 3018 A and 639 G,
   iii. 3018 A and 1248 T,
   iv. 4014 A and 201 G,
   v. 4014 A and 1329 A,
   vi. 4014 A and 1248 T,
   vii. 4014 A and 438 G,
   viii. 4014 A and 2805 T
   ix. 201 G and 2805 T,
   x. 201 G and 1248 T,
   xi. 201 G and 2460 T, and
   xii. 201 G and 3648 A;
b. a nucleotide sequence at least 90% identical to SEQ ID NO.: 1; and/or
c. a nucleotide sequence being at least 80% identical to SEQ ID NO. 1 and having in an alignment to the sequence depicted in SEQ ID NO. 1 at the following positions counting from the first nucleotide of the start codon one or more of the following nucleotides:
   i. 303 A,
   ii. 1029 A,
   iii. 1329 A, and
   iv. 2418 A.

For example, the following nucleotide positions are found:
i. 303 A, 1029 A, 1329 A, and 2418 A.

It was now found that the expression of a nucleotide sequence as described in the method of the invention in a plant cell results in much higher rates of indels compared to those seen in cells transformed with a control nucleic acid molecule. The control nucleic acid molecule is for example a nucleic acid molecule that is not codon optimized for the codon usage of wheat.

The use of an appropriate Cas9/gRNA expression construct and optimization of the culture period might be useful in developing the efficient targeted mutagenesis required to address the needs of plant science and molecular breeding in plants. The formed complex is capable of recognizing, binding to, and optionally nicking, unwinding, or cleaving all or part of said at least one target sequence.

The plant that is used in the method of the invention is for example a monocot plant, in particular a wheat plant.

Several polynucleotides that encode for a Cas9 endonuclease were tested in wheat plant cells. It was found that the sequences used in the method of the invention resulted in an improved efficiency, e.g. measured as cleavage activity or measured as knock out mutation efficiency, for example as shown in the examples.

Thus, in one embodiment, the polynucleotide used in the method of the invention comprises a codon-optimized Cas9 endonuclease encoding nucleic acid molecule with a nucleotide sequence that in an alignment with the nucleotide sequence depicted in SEQ ID NO 1 has counting from the first nucleotide of the start codon at the following positions at least one of the following nucleotide combinations:
i. 3018 A and 201 G,
ii. 3018 A and 639 G,
iii. 3018 A and 1248 T,
iv. 4014 A and 201 G,
v. 4014 A and 1329 A,
vi. 4014 A and 1248 T,
vii. 4014 A and 438 G,
viii. 4014 A and 2805 T
ix. 201 G and 2805 T,
x. 201 G and 1248 T,
xi. 201 G and 2460 T,
xii. 201 G and 3648 A,

For example, the following nucleotide positions are found:
i. 3018 A, 201 G, 639 G and 1248 T
ii. 4014 A and 201 G, 1329 A,1248 T,438 G, and 2805 T,
iii. 201 G and 2805 T, 1248 T, 2460 T, and 3648 A, and/or
iv. 3018 A, 201 G, 639 G, 1248 T, 4014 A, 1329 A, 438 G, and 2805 T, 2460 T, and 3648 A.

Further combinations that are unique to the polynucleotide that is used in the method of the invention can be found by comparing the nucleotide sequences of all known genes that encode a polypeptide that is 100%, 99%, 98%, 97%, 96%, 95%, 94%, 93%, 92%, 91% or 90% identical to the polypeptide as shown in SEQ ID No. 2, e.g. as described in the examples. Preferably, further unique positions are identified by comparing the nucleotide sequence of the polynucleotide as used in the method of the invention with a one that encodes the same polypeptide as shown in SEQ ID NO. 2.

It was found that these nucleotide combinations are unique for the optimized nucleic acid molecule resulting in an improved efficiency, e.g. measured as cleavage activity or measured as knock out mutation efficiency, for example as shown in the examples.

In a comparison of 183 polynucleotide sequences that all encode for the Cas9 endonuclease sequences as shown in SEQ ID NO. 2, it was found that the combinations shown in (i) to (xv) are unique to the polynucleotide sequences that is used in the method of the invention. The polynucleotide as used in the method of the invention results in an increased activity.

Accordingly, in one embodiment, more than one of these nucleotide combinations are present in the codon-optimized Cas9 endonuclease encoding nucleic acid molecule encoding for a polypeptide as shown in SEQ ID NO. 2.

E.g. 1, 2, 3, 4, 5, 6, 7, 8, 9, 10, 11, 12, 13, 14 or 15 of the above-mentioned combinations are present in the used polynucleotide. The combinations shown in (i) to (xv) can be present in the polynucleotide used in the method of the invention in combination with any of the other combinations in (i) to (xv), i.e. each combination can be combined with one or more of the other listed combinations. E.g. combinations (i), (ii), (iii), (iv), (v), (vi), (vii), (viii), (ix), (x), (xi) and/or (xii) can be combined with the combinations shown in ((i), (ii), (iii), (iv), (v), (vi), (vii), (viii), (ix), (x), (xi) and/or (xii). Accordingly, each of the combinations (i) to (xvi) can be combined with one or more or all of the combinations as shown in (i) to (xii), e.g. combinations (i) to (iii), are found in the sequence used in the method of the present invention or other combinations of (i) to (xii).

Though all the combinations are not explicitly listed here, any possible combination of (i) to (xii) shall be understood as being disclosed herein. It is without any doubt that the person skilled in the art will easily extract for the list above all possible options, an all can be used in combination with the method of the invention. In one embodiment, the nucleotide sequence comprises all of these above-mentioned combinations, e.g.
i. 3018 A and 201 G,
ii. 3018 A and 639 G,
iii. 3018 A and 1248 T,
iv. 4014 A and 201 G,
v. 4014 A and 1329 A,
vi. 4014 A and 1248 T,
vii. 4014 A and 438 G,
viii. 4014 A and 2805 T
ix. 201 G and 2805 T,
x. 201 G and 1248 T,
xi. 201 G and 2460 T,
xii. 201 G and 3648 A, and

For example, the following nucleotide positions are found:
xiii. 3018 A, 201 G, 639 G and 1248 T,
xiv. 4014 A and 201 G, 1329 A,1248 T,438 G, and 2805 T, and/or
xv. 201 G and 2805 T, 1248 T, 2460 T, and 3648 A, or
xvi. 3018 A, 201 G, 639 G, 1248 T, 4014 A, 1329 A, 438 G, and 2805 T, 2460 T, and 3648 A.

It was found that the expression of the nucleotide molecules as shown in SEQ ID NO. 1 results in an increased activity in wheat cells. The use of a polynucleotide comprising the sequence of SEQ ID NO. 1 showed an improved efficiency, e.g. an improved cellular cleavage efficiency, e.g. measured as cleavage activity or measured as knock out mutation efficiency in a cell, as for example shown in the examples. Thus, in one embodiment of the present invention, in the method of the invention the Cas9 endonuclease gene comprises a codon-optimized Cas9 endonuclease encoding polynucleotide of the invention with a nucleotide sequence 90% or more identical to SEQ ID NO.: 1. For example the codon-optimized Cas9 endonuclease encoding nucleic acid molecule is 90%, 91%, 92%, 93%, 94%, 95%, 96%, 97%, 98% or 99% or more identical to sequence shown in SEQ ID NO. 1. In one embodiment, the codon-optimized Cas9 endonuclease encoding nucleic acid molecule is 90%, 91%, 92%, 93%, 94%, 95%, 96%, 97%, 98% or 99% or more identical to sequence shown in SEQ ID NO. 1. The nucleotide sequence is for example 80%, 85%, 87%, 90%, 91%, 92%, 93%, 94%, 95%, 96%, 97%, 98% or 99% or more identical to SEQ ID No. 1 and encodes a Cas9 endonuclease having a Cas9 endonuclease sequence 80%, 85%, 87%, 90%, 91%, 92%, 93%, 94%, 95%, 96%, 97%, 98% or 99% or more identical to SEQ ID No. 2.

It was also found that the use of the nucleotides at the listed positions
i. 303 A,
ii. 1029 A,
iii. 1329 A, and/or
iv. 2418 A
   in the polynucleotide used in the method of the invention is advantageous in a nucleotide sequence being at least 80% identical to SEQ ID NO. 1 and having in an alignment to the sequence depicted in SEQ ID NO. 1 at the following positions counting from the first nucleotide of the start codon. E.g., the following nucleotide positions can be used:
v. 303 A, and 1029 A,
vi. 1329 A, and 2418 A
vii. 303 A, 1029A, and 1329 A,
viii. 1029A, 1329A, and 2418 A,
ix. 303 A, 1329 A, and 2418 A or
x. 303 A, 1029 A, 1329 A, and 2418 A

These nucleotides are unique to SEQ ID No. 1 if compared to other nucleotide sequences that are 80% or more identical to nucleotide sequence shown in SEQ ID NO. 1, for example as compared in the examples. SEQ ID No. 1 represents the nucleotide sequence that results in the highest efficiency if used in the method of the invention, e.g. in an improved cellular cleavage efficiency, e.g. measured as cleavage activity or measured as knock out mutation efficiency in a cell, for example as shown in the examples.

Thus, in the method of the present invention, a nucleotide sequence being at least 80% identical to SEQ ID NO. 1 and having in an alignment to the sequence depicted in SEQ ID NO. 1 counting from the first nucleotide of the start codon at the following positions one or more of the following nucleotides:
i. 303 A,
ii. 1029 A,
iii. 1329 A, and/or
iv. 2418 A can be used to encode a codon-optimized Cas9 and expressed in a wheat cell.

The nucleotide sequence is for example 80%, 85%, 87%, 90%, 91%, 92%, 93%, 94%, 95%, 96%, 97%, 98% or 99% or more identical to SEQ ID No. 1 and encodes a Cas9 endonuclease having a Cas9 endonuclease sequence 80%, 85%, 87%, 90%, 91%, 92%, 93%, 94%, 95%, 96%, 97%, 98% or 99% or more identical to SEQ ID No. 2.

In the method of the invention, preferably, a Cas9 endonuclease is used that is active in plant cells, e.g. in monocot plant cells, e.g. in wheat cells. Thus, in one embodiment, in the method of the invention, the polynucleotide used encodes for a Cas9 endonuclease comprising a polypeptide having a polypeptide sequence as shown in SEQ ID NO: 2, or a polypeptide sequence that is 90% or more identical to SEQ ID NO. 2, or a polypeptide encoded by a nucleotide sequence that is 91%, 92%, 93%, 94%, 95%, 96%, 97%, 98% or 99% or more identical to SEQ ID NO. 1.

For example, in the method of the invention, the polynucleotide used encodes for a Cas9 endonuclease comprising a polypeptide having a polypeptide sequence as shown in SEQ ID NO: 2, or a polypeptide sequence that is 90%, 91%, 92%, 93%, 94%, 95%, 96%, 97%, 98% or 99% or more identical to SEQ ID NO. 2, or a polypeptide encoded by a nucleotide sequence 90%, 91%, 92%, 93%, 94%, 95%, 96%, 97%, 98% or 99% or more identical to SEQ ID NO. 1.

In one embodiment, the polynucleotide used in the method of the invention has an Adenosine base at the following positions:

303 And 1029; 303 And 1329; 301A and 2418; 1029 and 1329; 1029 and 2418; 1329 and 2419; 303 And 1029 and 1329; 303 And 1029 and 2418; 1029 and 1329 and 2418; and/or 303 and 1029 and 1329 and 2418;
and is at least 80%, 85%, 87%, 90%, 91%, 92%, 93%, 94%, 95%, 96%, 97%, 98% or 99% identical to SEQ ID NO. 1, preferably encoding a polypeptide that is at least 90%, 91%, 92%, 93%, 94%, 95%, 96%, 97%, 98% or 99% identical to SEQ ID NO. 2.

The expression of the polynucleotide of the invention results in an improved efficiency of the Cas9 endonuclease in a cell. With improved efficiency or increased activity for the Cas9 endonuclease is meant that the Cas9 endonuclease has an improved cellular efficiency, e.g. the activity shown in the examples. For example, the expression of the polynucleotide used in the method of the invention results in an improved cellular cleavage efficiency, e.g. measured as cleavage activity or measured as knock out mutation efficiency in a cell or a compartment thereof, for example the expression of the polynucleotide as used in the invention results in higher rates of indels compared to those seen in cells transformed with a control. Preferably, the cell is a wheat cell.

The method of the invention further comprising selecting at least one cell that comprises the edited nucleotide sequence. Thus, the present invention relates to a method for modifying the genome of a plant cell, said method comprising providing to a plant cell comprising at least one target sequence to be modified, at least one non-naturally occurring guide RNA and at least one Cas9 endonuclease polynucleotide of the invention expressing a Cas9 endonuclease, wherein the guide RNA and Cas9 endonuclease encoded by the nucleotide by the invention are capable of forming a complex (PGEN), wherein said complex is capable of recognizing, binding to, and optionally nicking, unwinding, or cleaving all or part of said at least one target sequence; and, identifying at least one plant cell, wherein the at least one genome target sequence has been modified.

The polynucleotide used in the method of the invention shows an improved efficiency as described herein. Thus, the present invention also relates to the polynucleotide used in the method of the invention, for example to a polynucleotide molecule encoding a Cas9 endonuclease, wherein the nucleotide sequence of the polynucleotide molecule comprises a nucleotide sequence selected from the following nucleotide sequences:
a. a nucleotide sequence that in an alignment with the nucleotide sequence depicted in SEQ ID NO 1 has at the following positions counting from the first nucleotide of the start codon one or more of the following nucleotide combinations:
   i. 3018 A and 201 G,
   ii. 3018 A and 639 G,
   iii. 3018 A and 1248 T,
   iv. 4014 A and 201 G,
   v. 4014 A and 1329 A,
   vi. 4014 A and 1248 T,
   vii. 4014 A and 438 G,
   viii. 4014 A and 2805 T
   ix. 201 G and 2805 T,
   x. 201 G and 1248 T,
   xi. 201 G and 2460 T,
   xii. 201 G and 3648 A.

For example, the following nucleotide positions are found:
xiii. 3018 A, 201 G, 639 G and 1248 T,
xiv. 4014 A and 201 G, 1329 A,1248 T,438 G, and 2805 T, and/or
xv. 201 G and 2805 T, 1248 T, 2460 T, and 3648 A, or
xvi. 3018 A, 201 G, 639 G, 1248 T, 4014 A, 1329 A, 438 G, and 2805 T, 2460 T, and 3648 A
b. a nucleotide sequence at least 90% identical to SEQ ID NO.: 1, and/or
c. a nucleotide sequence being at least 80% identical to SEQ ID NO. 1 and having in an alignment to the sequence depicted in SEQ ID NO. 1 at the following positions counting from the first nucleotide of the start codon one or more of the following nucleotides:
   i. 303 A,
   ii. 1029 A,
   iii. 1329 A, and
   iv. 2418 A.

For example, the following nucleotide positions are found:
xi. 303 A, 1029 A, 1329 A, and 2418 A.

Thus, in one embodiment, the present invention relates to a Cas9 endonuclease gene comprises a codon-optimized Cas9 endonuclease encoding nucleic acid molecule with a nucleotide sequence that in an alignment with the nucleotide sequence depicted in SEQ ID NO 1 has counting from the first nucleotide of the start codon at the following positions at least one of the following nucleotide combinations:
i. 3018 A and 201 G,
ii. 3018 A and 639 G,
iii. 3018 A and 1248 T,
iv. 4014 A and 201 G,
v. 4014 A and 1329 A,
vi. 4014 A and 1248 T,
vii. 4014 A and 438 G,
viii. 4014 A and 2805 T
ix. 201 G and 2805 T,
x. 201 G and 1248 T,
xi. 201 G and 2460 T,
xii. 201 G and 3648 A, and

For example, the following nucleotide positions are found:
xiii. 3018 A, 201 G, 639 G and 1248 T,
xiv. 4014 A and 201 G, 1329 A,1248 T,438 G, and 2805 T, and/or
xv. 201 G and 2805 T, 1248 T, 2460 T, and 3648 A, or
xvi. 3018 A, 201 G, 639 G, 1248 T, 4014 A, 1329 A, 438 G, and 2805 T, 2460 T, and 3648 A

Further combinations that are unique to the polynucleotide of the invention can be found by comparing the nucleotide sequences of all known genes that encode a polypeptide that is 100%, 99%, 98%, 97%, 96%, 95%, 94%, 93%, 92%, 91% or 90% identical to the polypeptide as shown in SEQ ID No. 2, e.g. as described in the examples. Preferably, further unique positions are identified by comparing the nucleotide sequence of the polynucleotide of the invention with a one that encodes the same polypeptide as shown in SEQ ID NO. 2.

Accordingly, in one embodiment, more than one of these nucleotide combinations are present in the codon-optimized Cas9 endonuclease encoding nucleic acid molecule of the invention with a nucleotide sequence that in an alignment with the nucleotide sequence depicted in SEQ ID NO 1.

E.g. 1, 2, 3, 4, 5, 6, 7, 8, 9, 10, 11 or 12 of the above-mentioned combinations are present in the polynucleotide of the invention. The combinations shown in (i) to (xii) can be present in the polynucleotide of the invention in combination with any of the other combinations in (i) to (xii), i.e. each combination can be combined with one or more of the other listed combinations. E.g. combinations (i), (ii), (iii), (iv), (v), (vi), (vii), (viii), (ix), (x), (xi) and/or (xii) can be combined with the combinations shown in (i), (ii), (iii), (iv), (v), (vi), (vii), (viii), (ix), (x), (xi) and/or (xii) Accordingly, each of the combinations (i) to (xvi) can be combined with one or more or all of the combinations as shown in (i) to (xvi), e.g. combinations (i) to (iii), are found in the sequence used in the method of the present invention, or further combinations of (i) to (xii).

Though all the combinations are not explicitly listed here, any possible combination of (i) to (xvi) shall be understood as being disclosed herein. It is without any doubt that the person skilled in the art will easily extract for the list above all possible options, an all can be used in combination with the method of the invention. Finally, in one embodiment, the nucleotide sequence comprises all these above-mentioned combinations, e.g.
i. 3018 A and 201 G,
ii. 3018 A and 639 G,
iii. 3018 A and 1248 T,
iv. 4014 A and 201 G,
v. 4014 A and 1329 A,
vi. 4014 A and 1248 T,
vii. 4014 A and 438 G,
viii. 4014 A and 2805 T
ix. 201 G and 2805 T,
x. 201 G and 1248 T,
xi. 201 G and 2460 T,
xii. 201 G and 3648 A,
xiii. 3018 A, 201 G, 639 G and/or 1248 T,
xiv. 4014 A and 201 G, 1329 A,1248 T,438 G, and/or 2805 T, and
xv. 201 G and 2805 T, 1248 T, 2460 T, and/or 3648 A

For example, the following nucleotide positions are found:
xvi. 3018 A, 201 G, 639 G and 1248 T,
xvii. 4014 A and 201 G, 1329 A,1248 T,438 G, and 2805 T,
xviii. 201 G and 2805 T, 1248 T, 2460 T, and 3648 A, and/or
xix. 3018 A, 201 G, 639 G, 1248 T, 4014 A, 1329 A, 438 G, and 2805 T, 2460 T, and 3648 A

It was found that the expression of the nucleotide molecules of the invention, e.g. as shown in SEQ ID NO. 1 result in an increased activity in wheat cells. Surprisingly, the use of the polynucleotide of the invention comprising the sequence of SEQ ID NO. 1 showed the highest efficiency, e.g. an improved cellular cleavage efficiency, e.g. measured as cleavage activity or measured as knock out mutation efficiency in a cell, for example as shown in the examples.

Thus, the Cas9 endonuclease gene comprises a codon-optimized Cas9 endonuclease encoding nucleic acid molecule of the invention, e.g. polynucleotide with a nucleotide sequence 90% or more identical to SEQ ID NO.: 1. For example the codon-optimized Cas9 endonuclease encoding nucleic acid molecule of the invention is 90%, 91%, 92%, 93%, 94%, 95%, 96%, 97%, 98% or 99% or more identical to sequence shown in SEQ ID NO. 1. In one embodiment, the codon-optimized Cas9 endonuclease encoding nucleic acid molecule of the invention is 90%, 91%, 92%, 93%, 94%, 95%, 96%, 97%, 98% or 99% or more identical to sequence shown in SEQ ID NO. 1. The nucleotide sequence of the polynucleotide of the invention is for example 80%, 85%, 87%, 90%, 91%, 92%, 93%, 94%, 95%, 96%, 97%, 98% or 99% or more identical to SEQ ID No. 1 and encodes a Cas9 endonuclease having a Cas9 endonuclease sequence 80%, 85%, 87%, 90%, 91%, 92%, 93%, 94%, 95%, 96%, 97%, 98% or 99% or more identical to SEQ ID No. 2.

It was also found that the use of the nucleotides
i. 303 A,
ii. 1029 A,
iii. 1329 A, and, or
iv. 2418 A
   in the polynucleotide of the invention is advantageous. These nucleotides are unique to SEQ ID No. 1 if compared to other nucleotide sequences that are 80% or more identical to nucleotide sequence shown in SEQ ID NO. 1, for example as shown in in the examples. SEQ ID No. 1 represents the nucleotide sequence that results in the highest efficiency if used in the method of the invention, e.g. in an improved cellular cleavage efficiency, e.g. measured as cleavage activity or measured as knock out mutation efficiency in a cell, for example as shown in the examples.

Thus, the polynucleotide of the invention comprises a nucleotide sequence being at least 80% identical to SEQ ID NO. 1 and having in an alignment to the sequence depicted in SEQ ID NO. 1 counting from the first nucleotide of the start codon at the following positions one or more of the following nucleotides:
i. 303 A,
ii. 1029 A,
iii. 1329 A, and
iv. 2418 A can be used to encode the codon-optimized Cas9. The nucleotide sequence of the polynucleotide of the invention is for example 80%, 85%, 87%, 90%, 91%, 92%, 93%, 94%, 95%, 96%, 97%, 98% or 99% or more identical to SEQ ID No. 1 and encodes a Cas9 endonuclease having a Cas9 endonuclease sequence 80%, 85%, 87%, 90%, 91%, 92%, 93%, 94%, 95%, 96%, 97%, 98% or 99% or more identical to SEQ ID No. 2.

For example, the following nucleotide positions are found:
i. 303 A, 1029 A, 1329 A, and 2418 A.

The of the polynucleotide of the invention encodes a Cas9 endonuclease that is active in plant cells, e.g. in monocot plant cells, e.g. in wheat cells, like in wheat cells. Thus, in one embodiment, the polynucleotide of the invention used encodes for a Cas9 endonuclease comprising a polypeptide having a polypeptide sequence as shown in SEQ ID NO: 2, or a polypeptide sequence that is 90% or more identical to SEQ ID NO. 2, or a polypeptide encoded by a nucleotide sequence that is 91%, 92%, 93%, 94%, 95%, 96%, 97%, 98% or 99% or more identical to SEQ ID NO. 1.

For example, the polynucleotide of the invention encodes for a Cas9 endonuclease comprising a polypeptide having a polypeptide sequence as shown in SEQ ID NO: 2, or a polypeptide sequence that is 90%, 91%, 92%, 93%, 94%, 95%, 96%, 97%, 98% or 99% or more identical to SEQ ID NO. 2, or a polypeptide encoded by a nucleotide sequence 90%, 91%, 92%, 93%, 94%, 95%, 96%, 97%, 98% or 99% or more identical to SEQ ID NO. 1.

In one embodiment, the polynucleotide of the invention has an Adenosine base at the following positions:

303 and 1029; 303 and 1329; 303 and 2418; 1029 and 1329; 1029 and 2418; 1329 and 2419; 303 and 1029 and 1329; 303 and 1029 and 2418; 1029 and 1329 and 2418; and/or 303 and 1029 and 1329 and 2418;
and is at least 80%, 85%, 87%, 90%, 91%, 92%, 93%, 94%, 95%, 96%, 97%, 98% or 99% identical to SEQ ID NO. 1, preferably encoding a polypeptide that is at least 90%, 91%, 92%, 93%, 94%, 95%, 96%, 97%, 98% or 99% identical to SEQ ID NO. 2.

The modification at said target site is selected from the group consisting of (i) a replacement of at least one nucleotide, (ii) a deletion of at least one nucleotide, (iii) an insertion of at least one nucleotide, and (iv) any combination of (i)-(iii).

In one embodiment, the Cas9 endonuclease creates the modification in a coding region of the polynucleotide, in a regulatory region, e.g. a promoter region or an enhancer, or in a silence region, e.g. a region that does not affect the expression or activity of any gene or gene product.

The cellular expression of the polynucleotide of the invention or the polynucleotide used in the method of the invention results, for example, in at least one improved property, e.g. an improved property selected but not limited to properties from the group comprising an improved transformation efficiency, an improved mRNA stability, an improved expression efficiency, an improved translation efficiency, etc., e.g. when compared to a parent Cas9 endonuclease.

Recently, it was reported that the efficiency of the Cas9 endonuclease depends from the gRNA used. Thus, the polynucleotide of the present invention can also be used for the identification of a improved rRNA to form a complex with the Cas 9 endonuclease as shown in SEQ ID NO. 2, or a homolog thereof, e.g. a Cas9 endonuclease that is at least 90%, 91%, 92%, 93%, 94%, 95%, 96%, 97%, 98% or 99% identical to SEQ ID NO. 2. In one embodiment, not only one gRNA that can form an active complex with the Cas9 endonuclease is used in the method of the invention but more than one, e.g. several, like to 2, 5, 10, 20, 40, or more.

The method of the invention also allows modifying a target site in the genome of a plant cell and comprises providing one or more guide RNA and a donor nucleic acid (donor NA), e.g. a donor DNA, to a plant cell having a Cas9 endonuclease, wherein said guide RNA and Cas9 endonuclease are capable of forming a complex that enables the Cas9 endonuclease to introduce a double strand break at said target site, wherein the Cas9 endonuclease is expressed in the plant cell from the polynucleotide of the invention, e.g. the Cas9 endonuclease encoding sequence comprises the nucleotide sequence described above for use in the method of the invention or described as polynucleotide of the invention, and wherein said donor NA comprises a polynucleotide of interest.

For example, the donor NA comprises one or more nucleotide changes as compared to a corresponding endogenous unmodified genomic DNA. In one embodiment, the donor NA does not encode a full-length protein.

Further, the donor NA can comprise a heterologous regulatory element, e.g. the regulatory element comprises a promoter, or an enhancer element. The enhancer element can for example be plant derived. Further, in the heterologous regulatory element is for example selected from the group consisting of a regulatory element, 5′-UTR, intron, exon, coding sequence, and a promoter. The heterologous regulatory element can be e.g. from the same plant species as the polynucleotide involved resulting in improving one or more agronomic characteristics of the plant. The agronomic characteristics can for example be selected from the group consisting of abiotic stress tolerance, drought or nutrient deficiency, e.g. increase in yield or an increase in drought tolerance.

Accordingly, the method of the invention can comprise the following steps:
a) providing to a plant cell one or more guide RNA and a Cas9 endonuclease encoding polynucleotide, wherein said guide RNA and a Cas9 endonuclease expressed from said Cas 9 endonuclease encoding sequence of the invention are capable of forming a complex that enables the Cas9 endonuclease to introduce a double strand break at said target site; and,
b) identifying at least one plant cell that has a modification at said target, wherein the modification includes at least one deletion or substitution of one or more nucleotides in said target site;
   and wherein the Cas9 endonuclease encoding polynucleotide that comprises the polynucleotide sequence of the invention or described to be used in the method of the invention.

The plant cell is for example a monocot plant cell, e.g. a wheat plant cell.

In one embodiment, the guide RNA /Cas9 endonuclease complex is assembled in-vitro prior to being introduced into the cell as a ribonucleotide-protein complex.

the present invention relates to a plant, host cell, a plant cell, a plant organ, or a plant cell compartment comprising a recombinant DNA construct, said recombinant DNA construct comprising a promoter operably linked to a codon-optimized nucleotide sequence encoding a Cas9 endonuclease, wherein said Cas9 endonuclease is capable of binding to and creating a double strand break in a genomic target sequence said plant genome, and wherein the Cas 9 endonuclease encodes a sequence that comprises the polynucleotide sequence of the polynucleotide of the invention or used in the method of the invention.

Further, the present invention relates to a host cell, plant, plant organ, plant cell compartment or plant cell comprising a recombinant DNA construct and one or more guide RNA, wherein said recombinant DNA construct comprises a promoter operably linked to a codon-optimiued nucleotide sequence encoding a plant optimized Cas9 endonuclease, wherein said Cas9 endonuclease expressed from recombinant DNA construct and said guide RNA are capable of forming a complex and creating a double strand break in a genomic target sequence said plant genome and wherein the Cas9 endonuclease encoding sequence comprises the polynucleotide molecule sequence of the polynucleotide of the invention, and other as described herein. The host cell can be a prokaryotic cell or eukaryotic cell, for example, the host cell can be an E. coli or an agrobacterium sp. cell.

The promoter is for example a tissue specific promoter, e.g. a seed specific promoter, or a constitutive promoter. The recombinant DNA construct can comprise further regulatory elements, e.g. a terminator, enhancer, NEENA, and others as described herein. Thus, the present invention also relates to a host cell, a plant, a plant organ or a plant cell comprising the polypeptide of the invention or generated by the method of the invention or a progeny thereof, comprising the polynucleotide of the invention.

Accordingly, the present invention also relates to a recombinant DNA construct comprising a promoter operably linked to a nucleotide sequence encoding a plant optimized Cas9 endonuclease, wherein said Cas9 endonuclease is capable of binding to and creating a double strand break in a genomic target sequence in said plant genome, wherein the Cas9 endonuclease encoding sequence comprises the polynucleotide molecule sequence of the polynucleotide of the invention or used in the method of the invention.

Thus, for example, the recombinant DNA construct is an mRNA expression construct comprising: (i) the polynucleotide of the invention, and (ii) a second polynucleotide comprising a promoter active in plants, wherein the promoter is operably linked to the polynucleotide of the invention. Optionally, the construct comprises a gene or a fragment of a gene that is fused to the sequence encoding the Cas9 endonuclease and that results in the expression of a fusion Cas9 endonuclease as described herein.

Thus, the recombinant DNA construct comprises for example a promoter operably linked to a nucleotide sequence expressing a guide RNA, wherein said guide RNA is capable of forming a complex with a Cas9 endonuclease, and wherein said complex is capable of binding to and creating a double strand break in a genomic target sequence said plant genome, and wherein the Cas 9 endonuclease is expressed from a polynucleotide of the invention or used in the method of the invention.

The present invention also relates to a host cell, plant, plant organ or plant cell comprising the recombinant DNA construct of the invention. Accordingly, the present invention also relates to a recombinant host cell, plant, plant organ or plant cell. The recombinant host cell, plant, plant organ or plant cell can be a wheat cell, wheat plant, or a wheat organ.

Further, the present invention relates to a method for editing a nucleotide sequence in the genome of a cell, the method comprising providing a guide polynucleotide, a Cas9 endonuclease, and optionally a polynucleotide modification template, to a cell, wherein said guide RNA and Cas9 endonuclease are capable of forming a complex that enables the Cas9 endonuclease to introduce a double strand break at a target site in the genome of said cell, wherein said polynucleotide modification template comprises at least one nucleotide modification of said nucleotide sequence, and wherein the Cas 9 endonuclease is expressed from a polynucleotide that comprises the polynucleotide sequence of the polynucleotide of the invention.

According to the invention, the target side can have a length for example of between 12 and 30 nucleotides., Thus, the length of the target site can vary, and includes, for example, target sites that are at least 12, 13, 14, 15, 16, 17, 18, 19, 20, 21, 22, 23, 24, 25, 26, 27, 28, 29, 30 or more nucleotides in length. It is further possible that the target site can be palindromic. The nick/cleavage site can be within the target sequence or the nick/cleavage site could be outside of the target sequence. In another variation, the cleavage could occur at nucleotide positions immediately opposite each other to produce a blunt end cut or, in other cases, the incisions could be staggered to produce single-stranded overhangs, also called “sticky ends”, which can be either 5′ overhangs, or 3′ overhangs.

The nucleotide sequence in the genome of a cell can for example be selected from but not limited to the sequences of the group consisting of a promoter sequence, a terminator sequence, a regulatory element sequence, a splice site, a coding sequence, a polyubiquitination site, an intron site and an intron enhancing motif.

As the present invention provides a unique set of sequence positions that allows an improved cellular efficiency for the gene expressed in a plant cell, e.g. a monocot cell, for example a wheat cell, it is also advantageous to use the nucleotide sequence of the polynucleotide of the invention for the identification of further codon-optimized nucleotide sequences encoding a Cas9 endonuclease. The polynucleotide sequence can be aligned with other known and unknown sequences that encode for the polypeptide shown n SEQ ID No. 2. For examples, sequences that have at least as one of the herein mentioned advantageous nucleotides combinations can be tested for efficiency in other plants than wheat. Also, the sequence can be used to search for further codon-optimized sequences using software that proposes codon-optimized sequences for plants.

The polynucleotide of the invention further can comprise at least one operably linked nucleotide sequence encoding a nuclear localization signal (NLS). Thus, in one embodiment, in the polynucleotide of the invention, the nucleic acid sequence encoding the Cas9 endonuclease is operably linked to one or more NLS sequence, preferably one or more NLS sequence fused to the 5′ terminus and/or one or more NLS sequence fused to the 3′ terminus of the sequence encoding the CAS9 endonuclease. The first 51 nucleotide of SEQ ID NO. 3 show a NLS.

Thus, in one embodiment, the polynucleotide of the invention or the polynucleotide used in the method of the invention comprises the fusion of the polynucleotide of the invention, e.g. having a nucleic acid sequence as shown SEQ ID NO.: 1 or 90%, 91%, 92%, 93%, 94%, 95%, 96%, 97%, 98% or 99% or more identical thereto, with the NLS shown in SEQ ID NO. 3 and as explained in the examples, e.g. it is identical to a polynucleotide 90%, 91%, 92%, 93%, 94%, 95%, 96%, 97%, 98% or 99% or more identical to SEQ ID NO. 3. Thus, the present invention also relates to the polynucleotide used in the method of the invention, for example to a polynucleotide molecule encoding a Cas9 endonuclease, wherein the nucleotide sequence of the polynucleotide molecule comprises a nucleotide sequence of the invention or used in the method of the invention

In one embodiment of the present invention, the Cas9 endonuclease is a nickase. Accordingly, the polynucleotide molecule of the invention, the recombinant DNA construct of the invention or the polynucleotide used in the method of the invention have in the Cas9 polypeptide sequence depicted in SEQ ID NO. 2, a further mutation that results in a nickase activity, e.g. replacing the endonuclease activity. For example, after aligned with SEQ ID No. 2, the polypeptide has a D to A mutation at amino acid position 10, and/or a H to A amino acid mutation at position 840, if counted from the first codon.

In a further embodiment, the Cas9 endonuclease encoded by the polypeptide of the invention is a dead nuclease. Thus, for example the polypeptide as encoded by the polynucleotide of the invention has a R to A mutation at amino acid position 70, D to A mutation at amino acid position 10 and/or a H to A mutation at amino acid position 840, if counted from the first codon, or having one or more of the mutations as shown in Table A.

Further, the Cas9 endonuclease can have one or more of the following mutations as described in the database UniProtKB-Q99ZW2 (CAS9_STRP1):

TABLE A

| Position(s) | Description and Actions |
|---|---|
| 10 | D → A: Target DNA noncomplementary to the crRNA is not cleaved; nickase |
| | activity. Processes guide RNAs. In vivo, loss of Cas9-mediated CRISPR |
| | interference in plasmid transformation. Able to bind guide RNAs and target |
| | DNA but not cleave DNA; when associated with A-840. |
| 15 | S → A: Decreased DNA cleavage. |
| 66 | R → A: Significantly decreased DNA cleavage. |
| 70 | R → A: No DNA cleavage. |
| 74 | R → A: Significantly decreased DNA cleavage. |
| 78 | R → A: Moderately decreased DNA cleavage. |
| 97-150 | Missing: No nuclease activity. |
| 165 | R → A: Moderately decreased DNA cleavage. |
| 175-307 | Missing: About 50% nuclease activity. |
| 312-409 | Missing: No nuclease activity. |
| 475-477 | PWN → AAA: Slight decrease in target DNA cleavage and DNA-binding. Almost |
| | complete loss of DNA cleavage and binding; when associated with 1125-A--A- |
| | 1127. |
| 762 | E → A: Only cleaves 1 DNA strand, probably the noncomplementary strand. |
| | Processes guide RNAs correctly. In vivo, loss of Cas9-mediated CRISPR |
| | interference in plasmid transformation. |
| 840 | H → A: Target DNA complementary to the crRNA is not cleaved; nickase |
| | activity. In vivo, loss of Cas9-mediated CRISPR interference in plasmid |
| | transformation. Able to process and bind guide RNAs and target DNA but not |
| | cleave DNA; when associated with A-10. |
| 854 | N → A: Decreased DNA cleavage. Processes guide RNAs correctly. In vivo, |
| | retains Cas9-mediated CRISPR interference in plasmid transformation. |
| 863 | N → A: Only cleaves 1 DNA strand, probably the complementary strand. |
| | Processes guide RNAs correctly. In vivo, loss of Cas9-mediated CRISPR |
| | interference in plasmid transformation. |
| 982-983 | HH → AA: Processes guide RNAs correctly. |
| 982 | H → A: Decreased DNA cleavage. In vivo, loss of Cas9-mediated CRISPR |
| | interference in plasmid transformation. |
| 983 | H → A: Only cleaves 1 DNA strand, probably the noncomplementary strand. |
| 986 | D → A: Only cleaves 1 DNA strand, probably the noncomplementary strand. |
| | Processes guide RNAs correctly. In vivo, loss of Cas9-mediated CRISPR |
| | interference in plasmid transformation. |
| 1099-1368 | Missing: No nuclease activity. |
| 1125-1127 | DWD → AAA: No change in target DNA cleavage, slight decrease in DNA- |
| | binding. Almost complete loss of DNA cleavage and binding; when associated |
| | with 475-A--A-477. |
| 1132 | G → C: Probably inactivates protein. 1 Publication |
| 1333-1335 | RKR → AKA: Nearly complete loss of target DNA cleavage. |
| 1333 | R → A: Dramatically reduced target DNA binding, slightly decreased target |
| | cleavage. |
| 1335 | R → A: Dramatically reduced target DNA binding, slightly decreased target |
| | cleavage. |

The present invention also relates to a polynucleotide, a recombinant construct, or a polynucleotide used in the method of the invention that encodes a Cas9 nuclease that is active as endonuclease or nickase, or is inactive, and that is for example be fused to another polypeptide. In one embodiment, the Cas9 is a nickase or a dead nuclease and is fused to transcription activation or repression effectors, or epigenetic factors, e.g. such as histone-modifying/DNA methylation enzymes, or fluorescent proteins, e.g. for imaging of specific genomic loci, or a cytosine or adenine deaminases, e.g. for precisely altering DNA bases. For example, in the polynucleotide of the invention or the recombinant molecule of the invention or the construct of the invention, the coding region encodes a Cas9 nickase, fused to a reverse transcriptase.

Further, the present invention allows the selecting of a plant comprising an altered target site in its plant genome, the method comprises:

a) obtaining a first plant comprising at least one Cas9 endonuclease capable of introducing a double strand break at a target site in the plant genome; b) obtaining a second plant comprising one or more guide RNA that is capable of forming a complex with the Cas9 endonuclease of (a); c) crossing the first plant of (a) with the second plant of (b); d) evaluating the progeny of (c) for an alteration in the target site; and, e) selecting a progeny plant that possesses the desired alteration of said target site, whereby the Cas9 endonuclease is expressed from a gene comprising the polynucleotide of the invention.

Also, the present invention can be used to select a plant comprising an altered target site in its plant genome, the method comprising selecting at least one progeny plant that comprises an alteration at a target site in its plant genome, wherein said progeny plant was obtained by crossing a first plant comprising at least one a Cas9 endonuclease with a second plant comprising one or more guide RNA, wherein said Cas9 endonuclease is capable of introducing a double strand break at said target site and whereby the Cas9 endonuclease is expressed from a gene comprising the polynucleotide of the invention.

Thus, the method for selecting a plant of the invention also comprising selecting at least one progeny plant that comprises an alteration at a target site in its plant genome, wherein said progeny plant was obtained by crossing a first plant expressing at least one Cas9 endonuclease to a second plant comprising one or more guide RNA and a donor NA, wherein said Cas9 endonuclease is capable of introducing a double strand break at said target site, wherein said donor NA comprises a polynucleotide of interest, and whereby the Cas9 endonuclease is expressed from a gene comprising the polynucleotide of the invention.

Said guide polynucleotide (gRNA) can be a chimeric non-naturally occurring guide polynucleotide (gRNA) as described herein, and the guide polynucleotide/Cas endonuclease complex that comprises the gRNA can be capable of recognizing, binding to, and optionally nicking, unwinding, or cleaving all or part of a target sequence. In the method of the invention, the gene can be targeted with one or more guide polynucleotides (gRNAs), e.g. with multiple guide polynucleotides or gRNAs. It was found that targeting a gene with multiple gRNAs could improve the success rate of targeted mutagenesis and generate deletions to ensure gene knockout. (Li et al., Nat Biotechnol. 2013 Aug.; 31(8): 688-691.)

Further, the present invention relates to a composition comprising the polynucleotide of the invention, the construct of the invention or the plant, plant organ or plant cell of the invention, or a functional fragment thereof.

According to the invention, the Cas9 endonuclease can also be improved, e.g. by introducing at least one amino acid modification in a parent Cas9 endonuclease. Accordingly, for example the amino acid modification is located outside the RuVC and HNH domain of the parent Cas9 endonuclease, thereby creating an improved Cas9 endonuclease variant. The Cas9 endonuclease variant can show an improvement in at least one property when compared to said parent Cas9 endonuclease. The improved Cas9 endonuclease variant has at least one improved property selected from the group consisting of improved transformation efficiency and improved editing efficiency, when compared to said parent Cas9 endonuclease.

Further, the methods of the invention comprise that the Cas9 nuclease encoding polynucleotide is expressed in a plant or plant cell, e.g. wheat cell, or a cell compartment thereof, whereby the activity of the Cas9 is increased compared to a control. For example, the expression of the Cas9 endonuclease polynucleotide of claim 3 is increased in the plant cell or in a compartment thereof, compared to the use of a polynucleotide resulting in the expression of a control sequence.

Thus, the invention also relates to a polynucleotide molecule or the recombinant DNA construct of the invention that results in an increased Cas9 activity compared to a control sequence when expressed in a plant or plant cell, e,g. wheat cell, or in a compartment thereof, compared to a polynucleotide resulting in the expression of a control sequence.

It is to be understood that this invention is not limited to the particular methodology or protocols. It is also to be understood that the terminology used herein is for the purpose of describing particular embodiments only and is not intended to limit the scope of the present invention which will be limited only by the appended claims. It must be noted that as used herein and in the appended claims, the singular forms “a,” “and,” and “the” include plural reference unless the context clearly dictates otherwise. Thus, for example, reference to “a vector” is a reference to one or more vectors and includes equivalents thereof known to those skilled in the art, and so forth. The term “about” is used herein to mean approximately, roughly, around, or in the region of. When the term “about” is used in conjunction with a numerical range, it modifies that range by extending the boundaries above and below the numerical values set forth. In general, the term “about” is used herein to modify a numerical value above and below the stated value by a variance of 20 percent, preferably 10 percent up or down (higher or lower). As used herein, the word “or” means any one member of a particular list and also includes any combination of members of that list. The words “comprise,” “comprising,” “include,” “including,” and “includes” when used in this specification and in the following claims are intended to specify the presence of one or more stated features, integers, components, or steps, but they do not preclude the presence or addition of one or more other features, integers, components, steps, or groups thereof. The term “NA” when used herein means “nucleic acid” or “nucleic acids”. For clarity, certain terms used in the specification are defined and used as follows:

Coding region: As used herein the term “coding region” when used in reference to a structural gene refers to the nucleotide sequences which encode the amino acids found in the nascent polypeptide as a result of translation of a mRNA molecule. The coding region is bounded, in eukaryotes, on the 5′-side by the nucleotide triplet “ATG” which encodes the initiator methionine, prokaryotes also use the triplets “GTG” and “TTG” as start codon. On the 3′-side it is bounded by one of the three triplets which specify stop codons (i.e., TAA, TAG, TGA). In addition, a gene may include sequences located on both the 5′- and 3′-end of the sequences which are present on the RNA transcript. These sequences are referred to as “flanking” sequences or regions (these flanking sequences are located 5′ or 3′ to the non-translated sequences present on the mRNA transcript). The 5′-flanking region may contain regulatory sequences such as promoters and enhancers which control or influence the transcription of the gene. The 3′-flanking region may contain sequences which direct the termination of transcription, post-transcriptional cleavage and polyadenylation. A “coding region” of a nucleic acid is the portion of the nucleic acid or polypeptide, which is transcribed and translated in a sequence-specific manner to produce into a particular polypeptide or protein when placed under the control of appropriate regulatory sequences. The coding region is said to encode such a polypeptide or protein.

Complementary: “Complementary” or “complementarity” refers to two nucleotide sequences which comprise antiparallel nucleotide sequences capable of pairing with one another (by the base-pairing rules) upon formation of hydrogen bonds between the complementary base residues in the antiparallel nucleotide sequences. For example, the sequence 5′-AGT-3′ is complementary to the sequence 5′-ACT-3′. Complementarity can be “partial” or “total.” “Partial” complementarity is where one or more nucleic acid bases are not matched according to the base pairing rules. “Total” or “complete” complementarity between nucleic acid molecules is where each and every nucleic acid base is matched with another base under the base pairing rules. The degree of complementarity between nucleic acid molecule strands has significant effects on the efficiency and strength of hybridization between nucleic acid molecule strands. A “complement” of a nucleic acid sequence as used herein refers to a nucleotide sequence whose nucleic acid molecules show total complementarity to the nucleic acid molecules of the nucleic acid sequence.

Donor NA: the term “donor NA” or “doNA” means a nucleic acid, e.g. a DNA molecule, comprising two homology arms each comprising at least 15 bases complementary to two different areas of at least 15 consecutive bases of the target NA, wherein said two homology arms are directly adjacent to each other or are separated by one or more additional bases. The two different areas of the target NA to which the homology arms are complementary may be directly adjacent to each other or may be separated by additional bases of up to 20 kb, preferably up to 10 kb, preferably up to 5 kb, more preferably up to 3 kb, more preferably up to 2.5 kb, more preferably up to 2 kb. In the event a homology arm comprises more than 15 bases, it may be 100% complementary to the target NA or it may be at least 75% complementary, preferably at least 80% complementary, more preferably at least 85% complementary, more preferably at least 90% complementary, more preferably at least 95% complementary, more preferably at least 98% complementary to the target NA, wherein the homology arm comprises at least one stretch of at least 15 bases that are 100% complementary to a stretch of the same number of consecutive bases in the target NA, preferably the homology arm comprises at least one stretch of at least 18 bases that are 100% complementary to a stretch of the same number of consecutive bases in the target NA, more preferably the homology arm comprises at least one stretch of at least 20 bases that are 100% complementary to a stretch of the same number of consecutive bases in the target NA, even more preferably the homology arm comprises at least one stretch of at least 25 bases that are 100% complementary to a stretch of the same number of consecutive bases in the target NA, even more preferably the homology arm comprises at least one stretch of at least 50 bases that are 100% complementary to a stretch of the same number of consecutive bases in the target NA. The homology arms may have the same length and/or the same degree of complementarity to the target NA or may have different length and/or different degrees of complementarity to the target NA. The homology arms may be directly adjacent to each other or may be separated by a nucleic acid molecule comprising at least one base not present between the regions in the target nucleic acid complementary to the homology arms.

The term “directed evolution” is used synonymously with the term “metabolic evolution” herein and involves applying a selection pressure that favors the growth of mutants with the traits of interest. The selection pressure can be based on different culture conditions, ATP and growth coupled selection and redox related selection. The selection pressure can be carried out with batch fermentation with serial transferring inoculation or continuous culture with the same pressure.

Endogenous: An “endogenous” nucleotide sequence refers to a nucleotide sequence, which is present in the genome of a wild type microorganism.

Enhanced expression: “enhance” or “increase” the expression of a nucleic acid molecule in a microorganism are used equivalently herein and mean that the level of expression of a nucleic acid molecule in a microorganism is higher compared to a reference microorganism, for example a wild type. The terms “enhanced” or “increased” as used herein mean herein higher, preferably significantly higher expression of the nucleic acid molecule to be ex-pressed. As used herein, an “enhancement” or “increase” of the level of an agent such as a protein, mRNA or RNA means that the level is increased relative to a substantially identical microorganism grown under substantially identical conditions. As used herein, “enhancement” or “increase” of the level of an agent, such as for example a preRNA, mRNA, rRNA, tRNA, expressed by the target gene and/or of the protein product encoded by it, means that the level is increased 50% or more, for example 100% or more, preferably 200% or more, more preferably 5 fold or more, even more preferably 10 fold or more, most preferably 20 fold or more for example 50 fold relative to a suitable reference microorganism. The enhancement or increase can be determined by methods with which the skilled worker is familiar. Thus, the enhancement or increase of the nucleic acid or protein quantity can be deter-mined for example by an immunological detection of the protein. Moreover, techniques such as protein assay, fluorescence, Northern hybridization, densitometric measurement of nucleic acid concentration in a gel, nuclease protection assay, reverse transcription (quantitative RT-PCR), ELISA (enzyme-linked immunosorbent assay), Western blotting, radio-immunoassay (RIA) or other immunoassays and fluorescence-activated cell analysis (FACS) can be employed to measure a specific protein or RNA in a microorganism. De-pending on the type of the induced protein product, its activity or the effect on the phenotype of the microorganism may also be determined. Methods for determining the protein quantity are known to the skilled worker. Examples, which may be mentioned, are: the micro-Biuret method (Goa J (1953) Scand J Clin Lab Invest 5:218-222), the Folin-Ciocalteau method (Lowry O H et al. (1951) J Biol Chem 193:265-275) or measuring the absorption of CBB G-250 (Bradford M M (1976) Analyt Biochem 72:248-254).

The term “expression” or “gene expression” means the transcription of a specific gene(s) or specific genetic vector construct. The term “expression” or “gene expression” in particular means the transcription of gene(s) or genetic vector construct into mRNA. The process includes transcription of DNA and may include processing of the resulting RNA-product. The term “expression” or “gene expression” may also include the translation of the mRNA and therewith the synthesis of the encoded protein, i.e. protein expression. Thus, according to the present invention, the polynucleotide of the invention or the polynucleotide used in the method of the invention or the recombinant DNA construct is encoding a Cas9 endonuclease as described herein and its expression results in an increased efficiency or higher activity of the Cas9 endonuclease activity in the cell that comprises the polynucleotide of the invention compared to a cell that comprises a different nucleic acid encoding a different Cas9 endonuclease.

“Expression” also refers to the biosynthesis of a gene product, preferably to the transcription and/or translation of a nucleotide sequence, for example an endogenous gene or a heterologous gene, in a cell. For example, in the case of a structural gene, expression involves transcription of the structural gene into mRNA and—optionally—the subsequent translation of mRNA into one or more polypeptides. In other cases, expression may refer only to the transcription of the DNA harboring an RNA molecule.

Guide NA: the guide nucleic acid or guide NA or gNA or guide polynucleotide or gRNA comprises a spacer nucleic acid and a scaffold nucleic acid wherein the spacer NA and the scaffold NA are covalently linked to each other. In the event the scaffold NA consists of two molecules, the spacer NA is covalently linked to one molecule of the scaffold NA whereas the other molecule of the scaffold NA molecule hybridizes to the first scaffold NA molecule. Hence, a guide NA molecule may consist of one nucleic acid molecule or may consist of two nucleic acid molecules. Prefer-ably the guide NA consists of one molecule. Said guide polynucleotide (gRNA) can be a chimeric non-naturally occurring guide polynucleotide (gRNA) and the guide polynucleotide/Cas endonuclease complex that comprises the gRNA can be capable of recognizing, binding to, and optionally nicking, unwinding, or cleaving all or part of a target sequence. A gene can be targeted with one or more guide polynucleotides (gRNAs), e.g. with multiple guide polynucleotides or gRNAs.

Fusion NA: a fusion nucleic acid used in the method of the invention can comprise donor NA and guide NA, wherein the guide NA and the donor NA are covalently linked to each other. Further fusion polynucleotides are described herein, e.g. the fusion of the nucleic acid molecule encoding the Cas9 endonuclease or variant thereof, e.g. a nickase or a dead nickase, and other proteins.

Foreign: The term “foreign” refers to any nucleic acid molecule (e.g., gene sequence) which is introduced into a cell by experimental manipulations and may include sequences found in that cell as long as the introduced sequence contains some modification (e.g., a point mutation, the presence of a selectable marker gene, etc.) and is therefore different relative to the naturally-occurring sequence.

Functional fragment: the term “functional fragment” refers to any nucleic acid and/or pro-tein which comprises merely a part of the full length nucleic acid and/or full length poly-peptide of the invention but still provides the same function, i.e. the function of an AAT enzyme catalyzing the reaction of acryloyl-CoA and butanol to n-BA and CoA. Preferably, the fragment comprises at least 50%, at least 60%, at least 70%, at least 80%, at least 90% at least 95%, at least 98%, at least 99% of the sequence from which it is derived. Preferably, the functional fragment comprises contiguous nucleic acids or amino acids of the nucleic acid and/or protein from which the functional fragment is derived. A functional fragment of a nucleic acid molecule encoding a protein means a fragment of the nucleic acid molecule encoding a functional fragment of the protein.

Functional or operably linkage: The terms “functional linkage” or “functionally linked” is equivalent to the term “operable linkage” or “operably linked” and are to be understood as meaning, for example, the sequential arrangement of a regulatory element (e.g. a promoter) with a nucleic acid sequence to be expressed and, if appropriate, further regulatory elements (such as e.g., a terminator) in such a way that each of the regulatory elements can fulfill its intended function to allow, modify, facilitate or otherwise influence expression of said nucleic acid sequence. As a synonym the wording “operable linkage” or “operably linked” may be used. The expression may result depending on the arrangement of the nucleic acid sequences in relation to sense or antisense RNA. To this end, direct linkage in the chemical sense is not necessarily required. Genetic control sequences such as, for example, enhancer sequences, can also exert their function on the target sequence from positions which are further away, or indeed from other DNA molecules. Preferred arrangements are those in which the nucleic acid sequence to be expressed recombinantly is positioned behind the sequence acting as promoter, so that the two sequences are linked covalently to each other. In a preferred embodiment, the nucleic acid sequence to be transcribed is located behind the promoter in such a way that the transcription start is identical with the desired beginning of the chimeric RNA of the invention. Functional linkage, and an expression construct, can be generated by means of customary recombination and cloning techniques as described (e.g., Sambrook J, Fritsch E F and Maniatis T (1989); Silhavy et al. (1984) Experiments with Gene Fusions, Cold Spring Harbor Laboratory, Cold Spring Harbor (N.Y.); Ausubel et al. (1987) Current Protocols in Molecular Biology, Greene Publishing Assoc. and Wiley Inter-science; Gelvin et al. (Eds) (1990) Plant Molecular Biology Manual; Kluwer Academic Pub-lisher, Dordrecht, The Netherlands). However, further sequences, which, for example, act as a linker with specific cleavage sites for restriction enzymes, or as a signal peptide, may also be positioned between the two sequences. The insertion of sequences may also lead to the expression of fusion proteins. Preferably, the expression construct, consisting of a linkage of a regulatory region for example a promoter and nucleic acid sequence to be ex-pressed, can exist in a vector-integrated form or can be inserted into the genome, for ex-ample by transformation.

Gene: The term “gene” refers to a region operably linked to appropriate regulatory sequences capable of regulating the expression of the gene product (e.g., a polypeptide or a functional RNA) in some manner. A gene includes untranslated regulatory regions of DNA (e.g., promoters, enhancers, repressors, etc.) preceding (up-stream) and following (down-stream) the coding region (open reading frame, ORF). The term “structural gene” as used herein is intended to mean a DNA sequence that is transcribed into mRNA which is then translated into a sequence of amino acids characteristic of a specific polypeptide. The gene of the invention can for example comprise the polynucleotide of the invention.

Genome and genomic DNA: The terms “genome” or “genomic DNA” is referring to the heritable genetic information of a host organism. Said genomic DNA comprises the DNA of the nucleoid but also the DNA of the self-replicating plasmid.

Heterologous: The term “heterologous” with respect to a nucleic acid molecule or DNA refers to a nucleic acid molecule which is operably linked to, or is manipulated to become operably linked to, a second nucleic acid molecule to which it is not operably linked in nature, or to which it is operably linked at a different location in nature. A heterologous ex-pression construct comprising a nucleic acid molecule and one or more regulatory nucleic acid molecule (such as a promoter or a transcription termination signal) linked thereto for example is a constructs originating by experimental manipulations in which either a) said nucleic acid molecule, or b) said regulatory nucleic acid molecule or c) both (i.e. (a) and (b)) is not located in its natural (native) genetic environment or has been modified by experimental manipulations, an example of a modification being a substitution, addition, deletion, inversion or insertion of one or more nucleotide residues. Natural genetic environment refers to the natural genomic locus in the organism of origin, or to the presence in a genomic library. In the case of a genomic library, the natural genetic environment of the sequence of the nucleic acid molecule is preferably retained, at least in part. The environment flanks the nucleic acid sequence at least at one side and has a sequence of at least 50 bp, preferably at least 500 bp, especially preferably at least 1,000 bp, very especially preferably at least 5,000 bp, in length. A naturally occurring expression construct—for example the naturally occurring combination of a promoter with the corresponding gene—becomes a transgenic expression construct when it is modified by non-natural, synthetic “artificial” methods such as, for example, mutagenization. Such methods have been described (U.S. Pat. No. 5,565,350; WO 00/15815). For example, a protein encoding nucleic acid molecule operably linked to a promoter, which is not the native promoter of this molecule, is considered to be heterologous with respect to the promoter. Preferably, heterologous DNA is not endogenous to or not naturally associated with the cell into which it is introduced but has been obtained from another cell or has been synthesized. Heterologous DNA also includes an endogenous DNA sequence, which contains some modification, non-naturally occur-ring, multiple copies of an endogenous DNA sequence, or a DNA sequence which is not naturally associated with another DNA sequence physically linked thereto. Generally, although not necessarily, heterologous DNA encodes RNA or proteins that are not normally produced by the cell into which it is expressed. For example, a nucleic acid molecule sequence is “heterologous to” an organism or a second nucleic acid molecule sequence if it originates from a foreign or different species, or, if from the same species, is modified from its original form.

Hybridization: The term “hybridization” as used herein includes “any process by which a strand of nucleic acid molecule joins with a complementary strand through base pairing.” (J. Coombs (1994) Dictionary of Biotechnology, Stockton Press, New York). Hybridization and the strength of hybridization (i.e., the strength of the association between the nucleic acid molecules) is impacted by such factors as the degree of complementarity between the nucleic acid molecules, stringency of the conditions involved, the Tm of the formed hybrid, and the G:C ratio within the nucleic acid molecules. As used herein, the term “Tm” is used in reference to the “melting temperature.” The melting temperature is the temperature at which a population of double-stranded nucleic acid molecules becomes half dissociated into single strands. The equation for calculating the Tm of nucleic acid molecules is well known in the art. As indicated by standard references, a simple estimate of the Tm value may be calculated by the equation: Tm=81.5+0.41(% G+C), when a nucleic acid molecule is in aqueous solution at 1 M NaCl [see e.g., Anderson and Young, Quantitative Filter Hybridization, in Nucleic Acid Hybridization (1985)]. Other references include more sophisticated computations, which take structural as well as sequence characteristics into ac-count for the calculation of Tm. Stringent conditions, are known to those skilled in the art and can be found in Current Protocols in Molecular Biology, John Wiley & Sons, N.Y. (1989), 6.3.1-6.3.6.

Suitable hybridization conditions are for example hybridizing under conditions equivalent to hybridization in 7% sodium dodecyl sulfate (SDS), 0.5 M NaPO4, 1 mM EDTA at 50° C. with washing in 2×SSC, 0.1% SDS at 50° C. (low stringency) to a nucleic acid molecule comprising at least 50, preferably at least 100, more preferably at least 150, even more preferably at least 200, most preferably at least 250 consecutive nucleotides of the complement of a sequence. Other suitable hybridizing conditions are hybridization in 7% sodium dodecyl sulfate (SDS), 0.5 M NaPO4, 1 mM EDTA at 50° C. with washing in 1×SSC, 0.1% SDS at 50° C. (medium stringency) or 65° C. (high stringency) to a nucleic acid molecule comprising at least 50, preferably at least 100, more preferably at least 150, even more preferably at least 200, most preferably at least 250 consecutive nucleotides of a complement of a sequence. Other suitable hybridization conditions are hybridization in 7% sodium dodecyl sulfate (SDS), 0.5 M NaPO4, 1 mM EDTA at 50° C. with washing in 0.1×SSC, 0.1% SDS at 65° C. (very high stringency) to a nucleic acid molecule comprising at least 50, preferably at least 100, more preferably at least 150, even more preferably at least 200, most preferably at least 250 consecutive nucleotides of a complement of a sequence.

“Identity”: “Identity” when used in respect to the comparison of two or more nucleic acid or amino acid molecules means that the sequences of said molecules share a certain degree of sequence similarity, the sequences being partially identical.

For the determination of the percentage identity of two or more amino acids or of two or more nucleotide sequences several computer software programs have been developed. The identity of two or more sequences can be calculated with for example the software fasta, which presently has been used in the version fasta 3 (W. R. Pearson and D. J. Lipman, PNAS 85, 2444(1988); W. R. Pearson, Methods in Enzymology 183, 63 (1990); W. R. Pearson and D. J. Lipman, PNAS 85, 2444 (1988); W. R. Pearson, Enzymology 183, 63 (1990)). An-other useful program for the calculation of identities of different sequences is the standard blast program, which is included in the Biomax pedant software (Biomax, Munich, Federal Republic of Germany). This leads unfortunately sometimes to suboptimal results since blast does not always include complete sequences of the subject and the query. Neverthe-less as this program is very efficient it can be used for the comparison of a huge number of sequences. The following settings are typically used for such a comparison of sequences:

-p Program Name [String]; -d Database [String]; default=nr; -i Query File [File In]; de-fault=stdin; -e Expectation value (E) [Real]; default=10.0; -m alignment view options: 0=pairwise; 1=query-anchored showing identities; 2=query-anchored no identities; 3=flat query-anchored, show identities; 4=flat query-anchored, no identities; 5=query-anchored no identities and blunt ends; 6=flat query-anchored, no identities and blunt ends; 7=XML Blast output; 8=tabular; 9 tabular with comment lines [Integer]; default=0; -o BLAST report Output File [File Out] Optional; default=stdout; -F Filter query se-quence (DUST with blastn, SEG with others) [String]; default=T; -G Cost to open a gap (zero invokes default behavior) [Integer]; default=0; -E Cost to extend a gap (zero invokes default behavior) [Integer]; default=0; -X X dropoff value for gapped alignment (in bits) (zero invokes default behavior); blastn 30, megablast 20, tblastx 0, all others 15 [Integer]; default=0; -I Show GI's in deflines [T/F]; default=F; -q Penalty for a nucleotide mis-match (blastn only) [Integer]; default=−3; -r Reward for a nucleotide match (blastn only) [Integer]; default=1; -v Number of database sequences to show one-line descriptions for (V) [Integer]; default=500; -b Number of database sequence to show alignments for (B) [Integer]; default=250; -f Threshold for extending hits, default if zero; blastp 11, blastn 0, blastx 12, tblastn 13; tblastx 13, megablast 0 [Integer]; default=0; -g Perfom gapped alignment (not available with tblastx) [T/F]; default=T; -Q Query Genetic code to use [Integer]; default=1; -D DB Genetic code (for tblast[nx] only) [Integer]; default=1; -a Number of processors to use [Integer]; default=1; -O SeqAlign file [File Out] Optional; -J Believe the query defline [T/F]; default=F; -M Matrix [String]; default=BLOSUM62; -W Word size, default if zero (blastn 11, megablast 28, all others 3) [Integer]; default=0; -z Effective length of the database (use zero for the real size) [Real]; default=0; -K Number of best hits from a region to keep (off by default, if used a value of 100 is recommended) [Integer]; default=0; -P 0 for multiple hit, 1 for single hit [Integer]; default=0; -Y Effec-tive length of the search space (use zero for the real size) [Real]; default=0; -S Query strands to search against database (for blast[nx], and tblastx); 3 is both, 1 is top, 2 is bot-tom [Integer]; default=3; -T Produce HTML output [T/F]; default=F; -I Restrict search of database to list of GI's [String] Optional; -U Use lower case filtering of FASTA sequence [T/F] Optional; default=F; -y X dropoff value for ungapped extensions in bits (0.0 in-vokes default behavior); blastn 20, megablast 10, all others 7 [Real]; default=0.0; -Z X dropoff value for final gapped alignment in bits (0.0 invokes default behavior); blastn/megablast 50, tblastx 0, all others 25 [Integer]; default=0; -R PSI-TBLASTN checkpoint file [File In] Optional; -n MegaBlast search [T/F]; default=F; -L Location on query sequence [String] Optional; -A Multiple Hits window size, default if zero (blastn/megablast 0, all others 40 [Integer]; default=0; -w Frame shift penalty (OOF algo-rithm for blastx) [Integer]; default=0; -t Length of the largest intron allowed in tblastn for linking HSPs (0 disables linking) [Integer]; default=0.

Results of high quality are reached by using the algorithm of Needleman and Wunsch or Smith and Waterman. Therefore, programs based on said algorithms are preferred. Advantageously, the comparisons of sequences can be done with the program PileUp (J. Mol. Evolution., 25, 351 (1987), Higgins et al., CABIOS 5, 151 (1989)) or preferably with the programs “Gap” and “Needle”, which are both based on the algorithms of Needleman and Wunsch (J. Mol. Biol. 48; 443 (1970)), and “BestFit”, which is based on the algorithm of Smith and Waterman (Adv. Appl. Math. 2; 482 (1981)). “Gap” and “BestFit” are part of the GCG software-package (Genetics Computer Group, 575 Science Drive, Madison, Wis., USA 53711 (1991); Altschul et al., (Nucleic Acids Res. 25, 3389 (1997)), “Needle” is part of the The European Molecular Biology Open Software Suite (EMBOSS) (Trends in Genetics 16 (6), 276 (2000)). Therefore, preferably the calculations to determine the percentages of sequence identity are done with the programs “Gap” or “Needle” over the whole range of the sequences. The following standard adjustments for the comparison of nucleic acid sequences were used for “Needle”: matrix: EDNAFULL, Gap_penalty: 10.0, Extend_penalty: 0.5. The following standard adjustments for the comparison of nucleic acid sequences were used for “Gap”: gap weight: 50, length weight: 3, average match: 10.000, average mismatch: 0.000.

For example, a sequence, which is said to have 80% identity with sequence SEQ ID NO: 1 at the nucleic acid level is understood as meaning a sequence which, upon comparison with the sequence represented by SEQ ID NO: 1 by the above program “Needle” with the above parameter set, has an 80% identity. Preferably the identity is calculated on the complete length of the query sequence, for example SEQ ID NO: 1.

Isolated: The term “isolated” as used herein means that a material has been removed by the hand of man and exists apart from its original, native environment and is therefore not a product of nature. An isolated material or molecule (such as a DNA molecule or enzyme) may exist in a purified form or may exist in a non-native environment such as, for example, in a transgenic host cell. For example, a naturally occurring nucleic acid molecule or poly-peptide present in a living cell is not isolated, but the same nucleic acid molecule or poly-peptide, separated from some or all of the coexisting materials in the natural system, is isolated. Such nucleic acid molecules can be part of a vector and/or such nucleic acid molecules or polypeptides could be part of a composition and would be isolated in that such a vector or composition is not part of its original environment. Preferably, the term “isolated” when used in relation to a nucleic acid molecule, as in “an isolated nucleic acid sequence” refers to a nucleic acid sequence that is identified and separated from at least one contaminant nucleic acid molecule with which it is ordinarily associated in its natural source. Isolated nucleic acid molecule is nucleic acid molecule present in a form or setting that is different from that in which it is found in nature. In contrast, non-isolated nucleic acid molecules are nucleic acid molecules such as DNA and RNA, which are found in the state they exist in nature. For example, a given DNA sequence (e.g., a gene) is found on the host cell chromosome in proximity to neighboring genes; RNA sequences, such as a specific mRNA sequence encoding a specific protein, are found in the cell as a mixture with numerous other mRNAs, which encode a multitude of proteins. However, an isolated nucleic acid sequence comprising for example SEQ ID NO: 1 includes, by way of example, such nucleic acid sequences in cells which ordinarily contain SEQ ID NO: 1 where the nucleic acid sequence is in a genomic or plasmid location different from that of natural cells, or is other-wise flanked by a different nucleic acid sequence than that found in nature. The isolated nucleic acid sequence may be present in single- or double-stranded form. When an isolated nucleic acid sequence is to be utilized to express a protein, the nucleic acid sequence will contain at a minimum at least a portion of the sense or coding strand (i.e., the nucleic acid sequence may be single-stranded). Alternatively, it may contain both the sense and anti-sense strands (i.e., the nucleic acid sequence may be double-stranded). In one embodiment, the polynucleotide of the invention, the gene of the invention, the construct of the invention

Non-coding: The term “non-coding” refers to sequences of nucleic acid molecules that do not encode part or all of an expressed protein. Non-coding sequences include but are not limited enhancers, promoter regions, 3′ untranslated regions, and 5′ untranslated regions.

Polynucleotides, Nucleic acids and nucleotides: The terms “polynucleotides”, “nucleic acids” and “Nucleotides” refer to naturally occurring or synthetic or artificial nucleic acid or nucleotides. The terms “polynucleotides”, “nucleic acids” and “nucleotides” comprise deoxyribonucleotides or ribonucleotides or any nucleotide analogue and polymers or hybrids thereof in either single- or double-stranded, sense or antisense form. Unless otherwise indicated, a particular nucleic acid sequence also implicitly encompasses conservatively modified variants thereof (e.g., degenerate codon substitutions) and complementary sequences, as well as the sequence explicitly indicated. The terms “polynucleotides” and “nucleic acid” is used inter-changeably herein with “oligonucleotide,” and “nucleic acid molecule”. For example, “gene”, “cDNA, or “mRNA”, represent subgroups of the polynucleotides or nucleic acid molecules, characterized by further specific features well known in the art.

Nucleotide analogues: The term “nucleotide analogues” include nucleotides having modifications in the chemical structure of the bases, for example of the bases A, C, G, U or T, e.g., the modifications are at the sugar and/or phosphate, including, but not limited to, 5-position pyrimidine modifications, 8-position purine modifications, modifications at cytosine exocyclic amines, substitution of 5-bromo-uracil, and the like; and 2′-position sugar modifications, including but not limited to, sugar-modified ribonucleotides in which the 2′-OH is replaced by a group selected from H, OR, R, halo, SH, SR, NH2, NHR, NR2, or CN. Short hairpin RNAs (shRNAs) also can comprise non-natural elements such as non-natural bases, e.g., ionosin and xanthine, non-natural sugars, e.g., 2′-methoxy ribose, or non-natural phosphodiester linkages, e.g., methylphosphonates, phosphorothioates and peptides. In one embodiment, the polynucleotide of the invention refers to naturally occurring or synthetic or artificial nucleic acid molecules. In one embodiment, the polynucleotide comprises only natural occurring nucleotides, for example the polynucleotide does not comprise non-natural bases.

Nucleic acid sequence: The phrase “nucleic acid sequence” refers to a single- or double-stranded polymer of deoxyribonucleotide or ribonucleotide bases read from the 5′- to the 3′-end. It includes chromosomal DNA, self-replicating plasmids, infectious polymers of DNA or RNA and DNA or RNA that performs a primarily structural role. “Nucleic acid sequence” also refers to a consecutive list of abbreviations, letters, characters or words, which represent nucleotides. In one embodiment, a nucleic acid can be a “probe” which is a relatively short nucleic acid, usually less than 100 nucleotides in length. Often a nucleic acid probe is from about 50 nucleotides in length to about 10 nucleotides in length.

Oligonucleotide: The term “oligonucleotide” refers to an oligomer or polymer of ribonucleic acid (RNA) or deoxyribonucleic acid (DNA) or mimetics thereof, as well as oligonucleotides having non-naturally-occurring portions which function similarly. Such modified or substituted oligonucleotides are often preferred over native forms because of desirable properties such as, for example, enhanced cellular uptake, enhanced affinity for nucleic acid target and increased stability in the presence of nucleases. An oligonucleotide preferably includes two or more nucleomonomers covalently coupled to each other by linkages (e.g., phosphodiesters) or substitute linkages.

Overhang: An “overhang” is a relatively short single-stranded nucleotide sequence on the 5′- or 3′-hydroxyl end of a double-stranded oligonucleotide molecule (also referred to as an “extension,” “protruding end,” or “sticky end”).

Polypeptide: The terms “polypeptide”, “peptide”, “oligopeptide”, “polypeptide”, “gene prod-uct”, “expression product” and “protein” are used interchangeably herein to refer to a poly-mer or oligomer of consecutive amino acid residues.

Promoter: The terms “promoter”, or “promoter sequence” are equivalents and as used here-in, refer to a DNA sequence which when operably linked to a nucleotide sequence of inter-est is capable of controlling the transcription of the nucleotide sequence of interest into RNA. A promoter is located 5′ (i.e., upstream), proximal to the transcriptional start site of a nucleotide sequence of interest whose transcription into mRNA it controls, and provides a site for specific binding by RNA polymerase and other transcription factors for initiation of transcription. The promoter does not comprise coding regions or 5′ untranslated regions. The promoter may for example be heterologous or homologous to the respective cell. Suitable promoters can be derived from genes of the host cells where expression should occur or from pathogens for this host. In one embodiment, the recombinant DNA construct of the invention comprises the polynucleotide of the invention comprising a promoter sequence. The promoter is in one embodiment heterologous to the encoded Cas9 endonuclease. For example, a promoter operably linked to a heterologous coding sequence refers to a coding sequence from a species different from that from which the promoter was derived, or, if from the same species, a coding sequence which is not naturally associated with the promoter (e.g. a genetically engineered coding sequence or an allele from a different ecotype or variety).

The terms “production” or “productivity” are art-recognized and include the concentration of the fermentation product (for example, dsRNA) formed within a given time and a given fermentation volume (e.g., kg product per hour per liter). The term “efficiency of production” includes the time required for a particular level of production to be achieved (for example, how long it takes for the cell to attain a particular rate of output of a fine chemical).

Purified: As used herein, the term “purified” refers to molecules, either nucleic or amino acid sequences that are removed from their natural environment, isolated or separated. “Substantially purified” molecules are at least 60% free, preferably at least 75% free, and more preferably at least 90% free from other components with which they are naturally as-sociated. A purified nucleic acid sequence may be an isolated nucleic acid sequence. In one embodiment, the nucleic acid molecules as described herein, e.g. the polynucleotide of the invention or the recombinant DNA construct are purified.

The term “recombinant organism” or “recombinant cell” includes non-human organisms or cells which have been genetically modified such that they exhibit an altered or different genotype and/or phenotype (e. g., when the genetic modification affects coding nucleic acid sequences of the organism or cell) as compared to the wild type organism from which it was derived. A recombinant organism comprises at least one recombinant nucleic acid molecule. For example, the host cell, the plant, plant organ or plant cell of the invention are recombinant cells or recombinant organisms or comprise a recombinant organism or a recombinant cell.

The term “recombinant” with respect to nucleic acid molecules refers to nucleic acid molecules produced by man using recombinant nucleic acid techniques. The term comprises nucleic acid molecules which as such do not exist in nature or do not exist in the organism from which the nucleic acid molecule is derived, but are modified, changed, mutated or otherwise manipulated by man. Preferably, a “recombinant nucleic acid molecule” is a non-naturally occurring nucleic acid molecule that differs in sequence from a naturally occur-ring nucleic acid molecule by at least one nucleic acid. A “recombinant nucleic acid molecules” may also comprise a “recombinant construct” which comprises, preferably operably linked, a sequence of nucleic acid molecules not naturally occurring in that order. Preferred methods for producing said recombinant nucleic acid molecules may comprise cloning techniques, directed or non-directed mutagenesis, gene synthesis or recombination techniques. An example of such a recombinant nucleic acid molecule is a vector into which a heterologous DNA-sequence has been inserted or a gene or promoter which has been mutated compared to the gene or promoter from which the recombinant nucleic acid molecule derived. The mutation may be introduced by means of directed mutagenesis technologies known in the art or by random mutagenesis technologies such as chemical, UV light or x-ray mutagenesis or directed evolution technologies.

Significant increase: An increase for example in enzymatic activity, gene expression, productivity or yield of a certain product, that is larger than the margin of error inherent in the measurement technique, preferably an increase by about 10% or 25% preferably by 50% or 75%, more preferably 2-fold or-5 fold or greater of the activity, expression, productivity or yield of the control enzyme or expression in the control cell, productivity or yield of the control cell, even more preferably an increase by about 10-fold or greater. In one embodiment of the invention the increase of the activity or efficiency of the Cas9 if used according to the method of the invention is measured as cellular activity, as described in the examples. The cellular activity or efficiency of the polynucleotide of the invention used in the method of the invention is preferably 20%, 30%, 50%, 70%, 100%, 150%, 200%, 250% or 300% or higher compared to a control sequence, e.g. nucleic acid sequence encoding a Cas9 endonuclease that is less than 80% identical to SEQ ID NO. 1 and does not have at the following positions, counting from the first nucleotide of the start codon, one or more of the following nucleotides:
i. 303 A,
ii. 1029 A,
iii. 1329 A,
iv. and 2418 A

Significant decrease: A decrease for example in enzymatic activity, gene expression, productivity or yield of a certain product, that is larger than the margin of error inherent in the measurement technique, preferably a decrease by at least about 5% or 10%, preferably by at least about 20% or 25%, more preferably by at least about 50% or 75%, even more preferably by at least about 80% or 85%, most preferably by at least about 90%, 95%, 97%, 98% or 99%.

Spacer NA: the term “spacer nucleic acid” or “spacer NA” means a nucleic acid comprising at least 12 bases 100% complementary to the target NA. In the event the spacer NA comprises more than 12 bases, it may be at least 75% complementary to the target NA, preferably at least 80% complementary, more preferably at least 85% complementary, more preferably at least 90% complementary, more preferably at least 95% complementary, more preferably at least 98% complementary most preferably it is 100% complementary to the target NA, wherein the spacer NA comprises at least one stretch of at least 12 bases that are 100% complementary to a stretch of the same number of consecutive bases in the target NA, preferably the spacer NA comprises at least one stretch of at least 15 bases that are 100% complementary to a stretch of the same number of consecutive bases in the target NA, preferably the spacer NA comprises at least one stretch of at least 18 bases that are 100% complementary to a stretch of the same number of consecutive bases in the target NA, more preferably the spacer NA comprises at least one stretch of at least 20 bases that are 100% complementary to a stretch of the same number of consecutive bases in the target NA, even more preferably the spacer NA comprises at least one stretch of at least 25 bases that are 100% complementary to a stretch of the same number of consecutive bases in the target NA, even more preferably the spacer NA comprises at least one stretch of at least 50 bases that are 100% complementary to a stretch of the same number of consecutive bases in the target NA. The spacer NA is covalently linked to a scaffold NA. If the scaffold NA is consisting of two nucleic acid molecules, the spacer is covalently linked to one molecule of a scaffold NA.

Scaffold NA: the scaffold nucleic acid or scaffold NA comprises a nucleic acid forming a secondary structure comprising at least one hairpin, preferably at least two hairpins and/or a sequence that is/are bound by the site directed nucleic acid modifying polypeptide. Such site directed nucleic acid modifying polypeptides are known in the art, for example in WO/2014/150624; WO/2014/204728. The scaffold NA further comprises two regions each comprising at least eight bases being complementary to each other, hence capable to hybridize forming a double-stranded structure. If said regions of at least eight bases complementary to each other are comprising more than eight bases, each region comprises at least eight bases that are complementary to at least eight bases of the other region. The two complementary regions of the scaffold NA may be covalently linked to each other via a linker molecule forming a hairpin structure or may consist of two independent nucleic acid molecules.

Site directed nucleic acid modifying polypeptide: By “site directed nucleic acid modifying polypeptide” “nucleic acid-binding site directed nucleic acid modifying polypeptide” or “site directed polypeptide” it is meant a polypeptide that binds nucleic acids and is target-ed to a specific nucleic acid sequence. A site-directed nucleic acid modifying polypeptide as described herein is targeted to a specific nucleic acid sequence in the target nucleic acid either by mechanism intrinsic to the polypeptide or, preferably by the nucleic acid molecule to which it is bound. The nucleic acid molecule bound by the polypeptide comprises a sequence that is complementary to a target sequence within the target nucleic acid, thus targeting the bound polypeptide to a specific location within the target nucleic acid (the target sequence). Most site directed nucleic acid modifying polypeptides introduce dsDNA breaks, but they may be modified to have only nicking activity or the nuclease activity may be inactivated. The site directed nucleic acid modifying polypeptides may be bound to a further polypep-tide having an activity such as fluorescence or nuclease activity such as the nuclease activity of the FokI polypeptide or a homing endonuclease polypeptide such as I-SceI.

Substantially complementary: In its broadest sense, the term “substantially complementary”, when used herein with respect to a nucleotide sequence in relation to a reference or target nucleotide sequence, means a nucleotide sequence having a percentage of identity between the substantially complementary nucleotide sequence and the exact complementary sequence of said reference or target nucleotide sequence of at least 60%, more desirably at least 70%, more desirably at least 80% or 85%, preferably at least 90%, more preferably at least 93%, still more preferably at least 95% or 96%, yet still more preferably at least 97% or 98%, yet still more preferably at least 99% or most preferably 100% (the later being equivalent to the term “identical” in this context). Preferably identity is assessed over a length of at least 19 nucleotides, preferably at least 50 nucleotides, more preferably the entire length of the nucleic acid sequence to said reference sequence (if not specified other-wise below). Sequence comparisons are carried out using default GAP analysis with the University of Wisconsin GCG, SEQWEB application of GAP, based on the algorithm of Needleman and Wunsch (Needleman and Wunsch (1970) J Mol. Biol. 48: 443-453; as de-fined above). A nucleotide sequence “substantially complementary ” to a reference nucleotide sequence hybridizes to the reference nucleotide sequence under low stringency conditions, preferably medium stringency conditions, most preferably high stringency conditions (as defined above).

Target region: A “target region” of a nucleic acid is a portion of a nucleic acid that is identified to be of interest.

Transgene: The term “transgene” as used herein refers to any nucleic acid sequence, which is introduced into the genome of a cell by experimental manipulations. A transgene may be an “endogenous DNA sequence,” or a “heterologous DNA sequence” (i.e., “foreign DNA”). The term “endogenous DNA sequence” refers to a nucleotide sequence, which is naturally found in the cell into which it is introduced so long as it does not contain some modification (e.g., a point mutation, the presence of a selectable marker gene, etc.) relative to the naturally-occurring sequence.

Transgenic: The term “transgenic”—when referring to an organism—means the presence, as result of a transformation, for example a transient or stably transformation, of at least one recombinant nucleic acid molecule. The recombinant nucleic acid molecule comprises for example a nucleic acid sequence that is foreign or heterologous to the organism or the cell. The present invention also relates to the plant, plant cell or plant organism of the present invention, for example to a plant, plant cell or plant organism that is transgenic as result of the presence of the polynucleotide of the present invention or the recombinant DNA construct or a gene comprising the polynucleotide of the invention. For example, the plant, plant cell, plant organ or host cell can be transgenic as result of a transformation with the polynucleotide of the invention or the recombinant DNA construct of the invention or an expression construct that comprises a gene comprising the polynucleotide of the invention or can be a progeny of such a plant, plant cell or plant tissue or host cell if it is genetically modified compared to the plant, plant cell or host cell that was the wild type and being transformed.

Vector: As used herein, the term “vector” refers to a nucleic acid molecule capable of transporting another nucleic acid molecule to which it has been linked. One type of vector is a genomic integrated vector, or “integrated vector”, which can become integrated into the genomic DNA of the host cell. Another type of vector is an episomal vector, i.e., a plasmid or a nucleic acid molecule capable of extra-chromosomal replication. Vectors capable of directing the expression of genes to which they are operatively linked are referred to herein as “expression vectors”. In the present specification, “plasmid” and “vector” are used inter-changeably unless otherwise clear from the context. Thus, the present invention also relates to a vector comprising the polynucleotide of the invention or the recombinant DNA construct of the invention or a gene comprising the polynucleotide of the invention. The plant, plant cell, the plant organ or the host cell of the invention can be transformed by the vector or the invention.

Wild type: The term “wild type”, “natural” or “natural origin” means with respect to an organism or cell that said organism or cell is not changed, mutated, or otherwise manipulated by man. With respect to a polypeptide or nucleic acid sequence, that the polypeptide or nucleic acid sequence is naturally occurring or available in at least one naturally occurring organism or cell which is not changed, mutated, or otherwise manipulated by man. A wild type of a organism or cell refers to an organism or cell whose genome is present in a state as before the introduction of a genetic modification of a certain gene. The genetic modification may be e.g. a deletion of a gene or a part thereof or a point mutation or the introduction of a gene. The term “organism” means a non-human organism.

The term “yield” or “product/carbon yield” is art-recognized and includes the efficiency of the conversion of the carbon source into the product (i.e., fine chemical). This is generally written as, for example, kg product per kg carbon source. By increasing the yield or production of the compound, the quantity of recovered molecules or of useful recovered molecules of that compound in a given amount of culture over a given amount of time is increased.

This invention may, however, be embodied in many different forms and should not be construed as limited to the embodiments set forth herein; rather these embodiments are provided so that this disclosure will be thorough and complete and will fully convey the scope of the invention to those skilled in the art.

### FIGURE DESCRIPTION

FIG. 1: Schematic drawing of the ddPCR design indicating the location, orientation and sequences of the different probes and primers. The position of the predicted cut site by Cas9 and the R158Q edit are also indicated. The edit probe only binds to alleles modified by HDR, whereas the drop-off probe loses its binding site when insertions or deletions are created by NHEJ.

FIG. 2: Validation of the ddPCR assay by NGS analysis.

FIG. 3: ddPCR assay sensitivity established by serial dilution of HDR and NHEJ synthetic templates in a constant background (200 ng) of WT genomic DNA. The limit of detection was ˜0.1% for NHEJ and ˜0.04% for HDR, as determined by comparison with WT genomic DNA controls. Data are means ±standard deviation of two merged wells per dilution and three merged wells for the WT control.

FIG. 4: Impact of codon optimization on Cas9 activity in wheat protoplast cells. Cells were simultaneously transfected with a gRNA expression cassette targeting the wheat mlo gene and one of several Cas9 expression vectors. Samples were harvested 70 hours post transformation. Indel efficiencies were measured using a quantitative droplet digital PCR assay. Data are means ±standard deviation of two technical and two biological replicates.

### EXAMPLES

### Chemicals and Common Methods

Unless indicated otherwise, cloning procedures carried out for the purpose of the current invention including restriction digest, agarose gel electrophoresis, purification and ligation of nucleic acids, transformation, selection and cultivation of bacterial cells are performed as described (Sambrook J, Fritsch E F and Maniatis T (1989)). Sequence analysis of recombinant DNA is performed by LGC Genomics (Berlin, Germany) using the Sanger technology (Sanger et al., 1977). Unless described otherwise, chemicals and reagents are obtained from Sigma Aldrich (Sigma Aldrich, St. Louis, USA), from Promega (Madison, Wis., USA) or Bio-Rad Laboratories (Hercules, Calif., USA). Restriction endonucleases and Gibson assembly reagents are from New England Biolabs (Ipswich, Mass., USA). Oligonucleotides are synthesized by Integrated DNA Technologies (Coralville, Iowa, USA). Codon-optimized genes are from Genewiz (South Plainfield, N.J., USA).

### Example 1: Cas9 Coding Sequence Optimization

The original Cas9 gene was a codon-optimized version of the Streptococcus pyogenes Cas9 (SpCas9), constructed for expression in rice cells (Shan et al. (2013); Nature Biotech 31(8)). A second rice codon-optimized version, obtained using in-house GenEvolution Leto-1.7.23 software, was included in the experiments as well. To optimize the expression of Cas9 in wheat (Triticum aestivum) cells, we used GeneOptimizer, a BASF proprietary software tool. Different settings were tested with parameters set for codon usage for wheat high-expressing genes and optional removal of major cryptic splice sites. Alternatively, more stringent parameters were used for codon usage with only the most abundant wheat amino acid codons selected during optimization, followed by manual removal of major cryptic splice sites.

### Example 2: Plasmid Construction

The original Cas9 gene as well as the codon-optimized versions described above were tagged with a SV40 nuclear localization signal at the N-terminus and a Xenopus-derived Nucleoplasmin C nuclear localization signal at the C-terminus and synthesized. The synthesized genes were digested with Ncol and Nhel and cloned into a proprietary expression plasmid between the Ncol and Nhel sites. The resulting expression vectors include the maize polyubiquitin (Ubi) promoter for constitutive expression located upstream of the Cas9 gene and a fragment of the 3′ untranslated region of either the nopaline synthase gene of Agrobacterium tumefaciens or the 35S gene of Cauliflower mosaic virus at the 3′end.

Also, a gRNA expression cassette containing a chimeric guide RNA composed of a 20-bp protospacer site (Gil-Humanes et al. (2017); Plant J 89(6)) targeting the wheat mlo gene (Q94F71_WHEAT), a 76 bp guide RNA scaffold and the wheat polymerase III terminator sequence was synthesized. The recognition site of the gRNA is located on the antisense strand within exon 4 of the wheat mlo gene. The guide is specific for the 5A and 4D alleles of the mlo gene and shows one mismatch with the 4B allele at position 6 from the PAM sequence. Expression of the gRNA is driven by the polymerase III-type promoter of the wheat U6 snRNA gene. The synthesized cassette was cloned into a standard E. coli vector (pUC derivative) via EcoRV blunt end ligation.

All plasmids were transformed in E. coli for propagation and isolated using a ZymoPure II Plasmid Gigaprep kit for DNA purification (Zymo Research, Irvine, Calif., USA).

### Example 3: Design of a Droplet Digital PCR Assay to Simultaneously Identify Indel Mutations and Precise Edits

Introduction to Experimental Procedures

Current methods to detect genome editing events include gel-based systems, artificial reporter assays, high resolution melting curve analysis and next-generation sequencing. Droplet digital PCR is a rapid alternative to these methods enabling rapid and systematic quantification of genome editing outcomes at endogenous loci. In a droplet digital PCR system, each PCR sample is partitioned into many droplets. PCR amplification occurs simultaneously in each droplet. At the end of the run, each droplet is individually assessed for the presence (positive) or absence (negative) of a fluorescent signal. Using a Poisson statistical analysis, the ratio of positive to negative droplets yields absolute quantification of the initial number of copies of the target sequence.

To setup a ddPCR assay capable of simultaneously measuring NHEJ and HDR at endogenous loci, we designed three kinds of probes, all located within one amplicon. The first, a reference probe, is labeled with FAM and located away from the mutagenesis site. This probe counts all genomic copies of the target. The second, a so-called drop-off probe, is labeled with HEX and is located where the Cas9 nuclease cuts the mlo target. If Cas9 induces NHEJ, the drop-off probe loses its binding site, resulting in loss of HEX and leaving only the FAM signal of the reference probe. The third probe, also FAM-labeled, binds to the desired DNA edit, causing a gain of additional FAM signal when precise edits are introduced. With this assay, indel mutations, WT alleles and precise edits can be detected as distinct, clearly separated droplets with high sensitivity and low background signal.

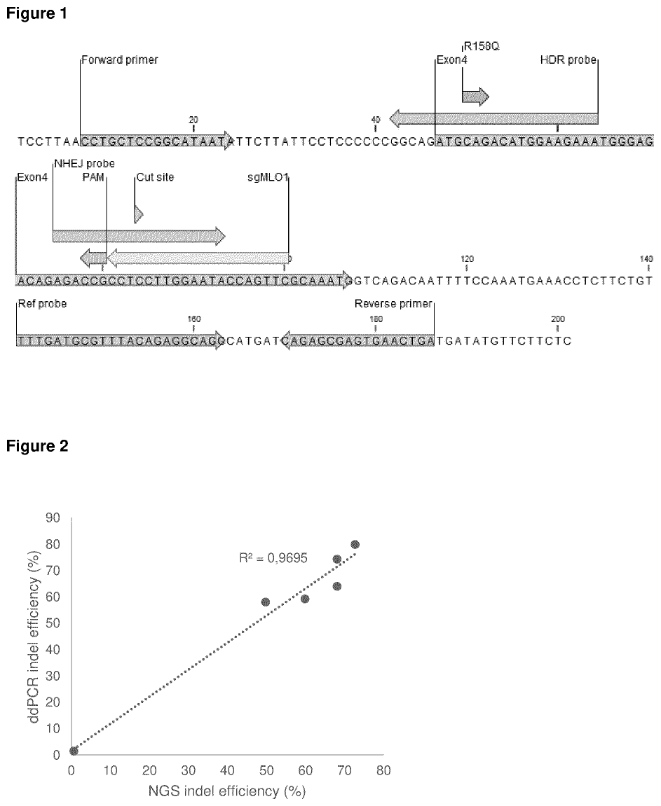

FIG. 1 shows the schematic drawing of the ddPCR design indicating the location, orientation and sequences of the different probes and primers. The position of the predicted cut site by Cas9 and the R158Q edit are also indicated. The edit probe only binds to alleles modified by HDR, whereas the drop-off probe loses its binding site when insertions or deletions are created by NHEJ.

Probes, Primers and gBLocks Design

ddPCR assays were designed using Primer3Plus software with modified settings compatible with the master mix: that is, 50 mM monovalent cations, 3.0 mM divalent cations, and 0 mM dNTPs with SantaLucia 1998 thermodynamic and salt correction parameters. The predicted nuclease cut site (3 bp upstream of PAM) was positioned mid-amplicon, with 70-100 bp flanking sequence either side up to the primer binding sites. To avoid loss of binding sites, primers and reference probe were designed away from the cut site. In addition, a dark, 3′-phosphorylated non-extendible oligonucleotide was designed to prevent the edit probe from binding to the WT sequence.

PCR primers were designed according to the following guidelines: primer length of 17-24 bases, primer melting temperature of 55 to 60° C. with an ideal temperature of 58° C., melting temperatures of the two primers differ by no more than 2° C., primer GC content of 35-65%, amplicon size of 100-250 bases.

Considerations for probe design were as follows: probes can bind to either strand of the target, probe GC content of 35-65%, no G at the 5′ end to prevent quenching of the 5′ fluorophore, melting temperature of the drop-off probe ranges from 61° C. to 64° C. with an ideal temperature of 62° C., length of the drop-off probe is less than 20 bases, melting temperatures of the reference and edit probe range from 63° C. to 67° C. with an ideal temperature of 65° C., length of the reference and edit probe of 20-24 bases. Preferably, probes should have a Tm 4-8° C. higher than the primers. Primer and probe designs were also screened for complementarity and secondary structure with the maximum ΔG value of any self-dimers, hairpins, and heterodimers set to −9.0 kcal/mole. All primers and probes were designed against the 5A allele of the wheat mlo gene.

The optimal annealing temperature was empirically determined using a temperature gradient PCR.

Synthetic dsDNA fragments (gBlocks, Integrated DNA Technologies) were used as positive controls for assay validation. HDR-positive controls contain the R158Q substitution at the desired edit site, whereas NHEJ-specific controls have a 1-bp insert at the predicted nuclease cut site. Lyophylized gBlocks were resuspended in 300 μl of TE and stored at min 20° C. Three additional dilutions in TE resulted in a master stock of approximately 600 copies/μl that was confirmed by ddPCR quantification. High-copy gBlock stocks were kept in a post-PCR environment to avoid contamination.

ddPCR Experiments and Quantification of Data

20×ddPCR mixes were composed of 18 μM forward and 18 μM reverse primers, 5 μM reference probe, 5 μM edit probe, 5 μM drop-off probe, and 10 μM dark probe . The following reagents were mixed in a 96-well plate to make a 25-μl reaction: 11 μl of ddPCR Supermix for Probes (no dUTP), 1.1 μl of 10× assay mix (BioRad Laboratories, Hercules, Calif., USA), 10 U of Hindlll-HF, 100-250 ng of genomic DNA in water, and water up to 22 μl.

Droplets were generated using a QX100 Droplet Generator according to the manufacturer's instructions (Bio-Rad Laboratories) and transferred to a 96-well plate for standard PCR on a C1000 Thermal cycler with a deep well block (BioRad Laboratories, Hercules, Calif., USA).

Thermal cycling consisted of a 10 min activation period at 95° C. followed by 40 cycles of a two-step thermal profile of 30 s at 95° C. denaturation and 3 min at 60° C. for combined annealing-extension and 1 cycle of 98° C. for 10 min.

After PCR, the droplets were analyzed using a QX100 Droplet Reader (BioRad Laboratories, Hercules, Calif., USA) in ‘absolute quantification’ mode. To enable proper gating for precise edits and indel events, experiments were performed using both negative and positive controls (non-modified genomic DNA and gBlocks containing the R158Q mutation, respectively). In two-dimensional plots, droplets without templates were gated as negative population. Droplets containing only NHEJ (FAM+, HEX−), only HDR alleles (FAM++, HEX−) or only WT alleles (FAM+, HEX+) were manually gated as separate populations. Allelic frequencies were quantified using the QuantaSoft v.1.2.10.0 software (BioRad Laboratories, Hercules, Calif., USA).

Validation of ddPCR by Amplicon Deep Sequencing and Determination of Detection Limit

The designed ddPCR assay was verified by next-generation sequencing (NGS) of the target region using a pair of primers specific for the A subgenome copy of the wheat mlo gene (Seq ID NO: 17/Seq ID NO: 18). The obtained amplicons were purified and subjected to deep-sequencing (2×250 bp paired ends) by Genewiz Inc using an Illumina MiSeq System. A very good correlation (R²=0.96) was observed between the indel allele frequencies detected by ddPCR and NGS across different samples, demonstrating the sensitivity and reliability of the ddPCR assay. In FIG. 2 the validation of the ddPCR assay by NGS analysis is shown.

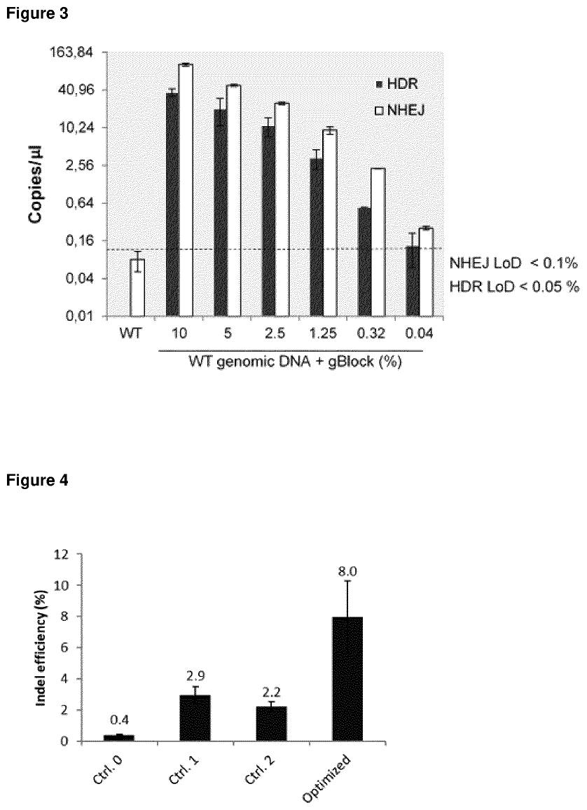

To calculate the ddPCR assay's limit of detection, we spiked wild-type genomic wheat DNA with different amounts of the HDR- and NHEJ-specific gBlocks (Seq ID NO: 19/Seq ID NO: 20) and found that the assay was reproducible and linear over a wide range of input DNA. The limit of detection was approximately 0.1% for NHEJ and well below 0.04 for % HDR alleles. This indicates that at least one indel or precise edit event from 1,000 copies of the genome can be captured by the assay. The ddPCR assay sensitivity established by serial dilution of HDR and NHEJ synthetic templates in a constant background (200 ng) of WT genomic DNA is shown in FIG. 3.

### Example 4: Wheat Protoplast Transfection

Transformation of wheat protoplast cells was performed as described by Wang et al. (2014) Nature; 32(9) with minor modifications. Protoplasts were isolated from the youngest fully developed leaf of 10-day-old aseptically grown wheat seedlings. Healthy leaves are bundled in stacks of five and cut into fine strips with a sharp razor blade. The strips are then infiltrated with cell wall-dissolving enzyme solution (1.5% cellulase R10 and 0.75% macerozyme R10 in 10 mM KCl and 0.6 M mannitol, pH 7.5) and incubated overnight in the dark with gentle shaking (40 rpm) at 24° C. After enzymatic digestion, the released protoplasts are collected by filtering the mixture through 40-μm nylon meshes and resuspended in W5 solution (Wang et al. (2014) Nature; 32(9)). The resuspended protoplasts are kept on ice and allowed to settle by gravity, after which the cell pellet is suspended in MMG solution (Wang et al. (2014) Nature;32(9)). For transformation, 200 μl of cells (2.5×10⁵) are mixed with 20 μg plasmid DNA and 220 μl of freshly prepared polyethylene glycol (PEG) solution. The mixture is incubated for 15-20 min in the dark. After removing the PEG solution, the transformed protoplasts are transferred into six-well plates and incubated at 24° C. for at least 48 h. Finally, the protoplasts are collected by centrifuging at 12,000 rpm for 1 min at room temperature.

### Example 5: Wheat Codon-Optimized Cas9 Shows Enhanced Indel Efficiency

To test the effectiveness of the codon-optimized Cas9 version, wheat protoplast cells were co-transfected with the CRISPR editing tools (Cas9 expression vectors, gRNA expression cassette) as described above. Transformed protoplasts were harvested 60 hours after transfection and indel formation was analyzed by ddPCR. As expected, very low levels of indels were found in negative controls, that is cells transfected with a GFP reporter plasmid (Ctrl. 0). Interestingly, transfecting cells with Cas9 codon-optimized for expression in wheat (‘Optimized’) resulted in much higher rates of indels compared to those seen in cells transformed with the rice-optimized Cas9 versions (Ctrl. 1 and Ctrl. 2). Pooled over two independent experiments, the wheat Cas9 optimized version showed a 2.75-fold increase in indel efficiency relative to the original gene (Ctrl .1) used for generation of stably edited wheat in Wang et al. (2014; Nature Biotech 32(9). The Impact of codon optimization on Cas9 activity in wheat protoplast cells is shown in FIG. 4.

1. A method for modifying a target site in the genome of a plant cell, the method comprising providing one or more guide RNA and a Cas endonuclease to said plant cell, wherein said guide RNA and Cas endonuclease are capable of forming a complex that enables the Cas endonuclease to introduce a double strand break at said target site, and wherein the Cas9 endonuclease is expressed in the plant cell from a polynucleotide comprising a codon-optimized Cas9 endonuclease encoding nucleic acid molecule with a nucleotide sequence selected from the following nucleotide sequences:
a. a nucleotide sequence that in an alignment with the nucleotide sequence depicted in SEQ ID NO. 1 has at the following positions counting from the first nucleotide of the start codon one or more of the following nucleotide combinations:
i. 3018 A and 201 G,
ii. 3018 A and 639 G,
iii. 3018 A and 1248 T,
iv. 4014 A and 201 G,
v. 4014 A and 1329 A,
vi. 4014 A and 1248 T,
vii. 4014 A and 438 G,
viii. 4014 A and 2805 T
ix. 201 G and 2805 T,
x. 201 G and 1248 T,
xi. 201 G and 2460 T, and/or
xii. 201 G and 3648 A,
b. a nucleotide sequence at least 90% identical to SEQ ID NO.: 1; and/or
c. a nucleotide sequence being at least 80% identical to SEQ ID NO. 1 and having in an alignment to the sequence depicted in SEQ ID NO. 1 at the following positions counting from the first nucleotide of the start codon one or more of the following nucleotides:
i. 303 A,
ii. 1029 A,
iii. 1329 A, and/or
iv. 2418 A.

2. The method of claim 1, wherein the nucleotide sequence that in an alignment with the nucleotide sequence depicted in SEQ ID NO. 1 has at the following positions counting from the first nucleotide of the start codon one or more of the following nucleotide combinations:
i. 3018 A, 201 G, 639 G and 1248 T,
ii. 4014 A and 201 G, 1329 A,1248 T,438 G, and 2805 T,
iii. 201 G and 2805 T, 1248 T, 2460 T, and 3648 A, and/or
iv. 3018 A, 201 G, 639 G, 1248 T, 4014 A, 1329 A, 438 G, and 2805 T, 2460 T, and 3648 A

3. The method of claim 1, wherein the plant is a wheat plant.

4. The method of claim 1, wherein the Cas9 endonuclease comprises the polypeptide sequence as shown in SEQ ID NO: 2, or a polypeptide sequence 90% or more identical to SEQ ID NO. 2, or a polypeptide encoded by a nucleotide sequence 90% or more identical to SEQ ID NO. 1.

5. A polynucleotide molecule encoding a Cas9 endonuclease, wherein the nucleotide sequence of the polynucleotide molecule comprises a nucleotide sequence selected from the following nucleotide sequences:
a. a nucleotide sequence that in an alignment with the nucleotide sequence depicted in SEQ ID NO. 1 has at the following positions counting from the first nucleotide of the start codon one or more of the following nucleotide combinations:
i. 3018 A and 201 G,
ii. 3018 A and 639 G,
iii. 3018 A and 1248 T,
iv. 4014 A and 201 G,
v. 4014 A and 1329 A,
vi. 4014 A and 1248 T,
vii. 4014 A and 438 G,
viii. 4014 A and 2805 T
ix. 201 G and 2805 T,
x. 201 G and 1248 T,
xi. 201 G and 2460 T, and/or
xii. 201 G and 3648 A;
b. a nucleotide sequence at least 90% identical to SEQ ID NO.: 1; and/or
c. a nucleotide sequence being at least 80% identical to SEQ ID NO. 1 and having in an alignment to the sequence depicted in SEQ ID NO. 1 at the following positions counting from the first nucleotide of the start codon one or more of the following nucleotides:
i. 301 A,
ii. 1029 A,
iii. 1329 A, and/or
iv. 2418 A.

6. The polynucleotide of claim 5, wherein the nucleotide sequence that in an alignment with the nucleotide sequence depicted in SEQ ID NO. 1 has at the following positions counting from the first nucleotide of the start codon one or more of the following nucleotide combinations:
i. 3018 A, 201 G, 639 G and 1248 T,
ii. 4014 A and 201 G, 1329 A,1248 T,438 G, and 2805 T,
iii. 201 G and 2805 T, 1248 T, 2460 T, and 3648 A, and/or
iv. 3018 A, 201 G, 639 G, 1248 T, 4014 A, 1329 A, 438 G, and 2805 T, 2460 T, and 3648 A.

7. A method for modifying a target site in the genome of a plant cell, the method comprising providing one or more guide RNAs and a donor DNA to a plant cell having a Cas9 endonuclease, wherein said guide RNA and Cas9 endonuclease are capable of forming a complex that enables the Cas9 endonuclease to introduce a double strand break at said target site, wherein the Cas9 endonuclease is expressed in the plant cell from a polynucleotide comprising the polynucleotide of claim 5, and wherein said donor DNA comprises a polynucleotide of interest.

8. A method for modifying a target site in the genome of a wheat cell, the method comprising:
a) providing to a wheat cell one or more guide RNA and a Cas 9 endonuclease encoding sequence, wherein said guide RNA and a Cas 9 endonuclease expressed from said Cas 9 endonuclease encoding sequence are capable of forming a complex that enables the Cas endonuclease to introduce a double strand break at said target site; and,
b) identifying at least one wheat cell that has a modification at said target, wherein the modification includes at least one deletion or substitution of one or more nucleotides in said target site;
and wherein the Cas 9 endonuclease encoding sequence that comprises the polynucleotide sequence of the polynucleotide of claim 5.

9. A plant, host cell, a plant cell, a plant organ, or a plant cell compartment comprising a recombinant DNA construct, said recombinant DNA construct comprising a promoter operably linked to a codon-optimized nucleotide sequence encoding a Cas9 endonuclease, wherein said Cas9 endonuclease is capable of binding to and creating a double strand break in a genomic target sequence said plant genome, and wherein the Cas 9 endonuclease encoding sequence that comprises the polynucleotide sequence of the polynucleotide of claim 5.

10. A plant, a host cell, a plant cell, a plant organ, or a plant cell compartment comprising a recombinant DNA construct and one or more guide RNA, wherein said recombinant DNA construct comprises a promoter operably linked to a codon-optimized nucleotide sequence encoding a Cas9 endonuclease, wherein said Cas9 endonuclease and guide RNA are capable of forming a complex and creating a double strand break in a genomic target sequence in said plant genome and wherein the Cas 9 endonuclease encoding sequence that comprises the polynucleotide sequence of the polynucleotide of claim 5.

11. A recombinant DNA construct comprising a promoter operably linked to a codon-optimized nucleotide sequence encoding a Cas9 endonuclease, wherein said Cas9 endonuclease is capable of binding to and creating a double strand break in a genomic target sequence of said plant genome, wherein the Cas 9 endonuclease encoding sequence comprising the polynucleotide sequence of the polynucleotide of claim 5.

12. A recombinant DNA construct comprising a promoter operably linked to a nucleotide sequence expressing a guide RNA, wherein said guide RNA is capable of forming a complex with a Cas9 endonuclease, and wherein said complex is capable of binding to and creating a double strand break in a genomic target sequence said plant genome, and wherein the Cas 9 endonuclease is expressed from a polynucleotide that comprises the polynucleotide sequence of the polynucleotide of claim 5.

13. A method for editing a nucleotide sequence in the genome of a cell, the method comprising providing one or more guide RNA, a Cas endonuclease, and optionally a polynucleotide modification template, to a cell, wherein said guide RNA and Cas endonuclease are capable of forming a complex that enables the Cas endonuclease to introduce a double strand break at a target site in the genome of said cell, wherein said polynucleotide modification template comprises at least one nucleotide modification of said nucleotide sequence, and wherein the Cas 9 endonuclease is expressed from a polynucleotide that comprises the polynucleotide sequence of the polynucleotide of claim 5.

14. The method of claim 13, wherein the nucleotide sequence in the genome of a cell is selected from the group consisting of a promoter sequence, a terminator sequence, a regulatory element sequence, a splice site, a coding sequence, a polyubiquitination site, an intron site and an intron enhancing motif.

15. (canceled)

16. The polynucleotide molecule of claim 5, wherein the nucleotide sequence molecule encoding the Cas9 endonuclease further comprises one or more NLS sequence.

17. The polynucleotide molecule of claim 5, wherein the Cas9 nuclease is a nickase having if aligned with the Cas9 polypeptide sequence depicted in SEQ ID NO. 2, and/or a D to A mutation at amino acid position 10 and/or a H to A amino acid mutation at position 840, or is a dead nuclease having a R to A mutation at amino acid position 70, and/or a D to A mutation at amino acid position 10 and/or a H to A mutation at amino acid position 840, or having one or more of the mutations as shown in FIG. 1.

18. The polynucleotide molecule of claim 5, wherein the Cas9 nuclease is active or inactive, and is fused to another polypeptide.

19. The polynucleotide molecule of claim 5, wherein the Cas9 nuclease is inactive, and is fused to transcription activation or repression effectors, epigenetic factors, such as histone-modifying/DNA methylation enzymes, fluorescent proteins for imaging of specific genomic loci, cytosine or adenine deaminases for precisely altering DNA bases.

20. The polynucleotide molecule of claim 5, wherein the Cas9 nuclease is a nickase and is fused to a reverse transcriptase.

21. The polynucleotide molecule of claim 16, wherein the one or more NLS sequence is fused to the 5′ terminus and/or is fused to the 3′ terminus of the sequence encoding the Cas9 endonuclease.
